(19) United States
(12) Patent Application Publication
(10) Pub. No.: US 2016/0246934 A1
(43) Pub. Date: Aug. 25, 2016

(54) System and Method for Health Assessment of Animals

(71) Applicant: IVET PTY LTD, East Bendigo, Victoria (AU)

(72) Inventor: Robert Hugo Dunlop, East Bendigo (AU)

(73) Assignee: IVET PTY LTD, East Bendigo, Victoria (AU)

(21) Appl. No.: 14/912,875

(22) PCT Filed: Aug. 19, 2014
(86) PCT No.: PCT/AU2014/000819

§ 371 Date: Feb. 18, 2016

(30) Foreign Application Priority Data
Aug. 19, 2013 (AU) 2013903134

(51) Int. Cl. G06F 19/00; H04L 29/08; A01K 29/00

(52) U.S. Cl. CPC G06F 19/3406; A01K 29/005; G06F 19/3425; H04L 67/32

(57) ABSTRACT

A method for remotely managing the health of animals, the method including: receiving measurement data relating to the health or treatment of one or more animals in one or more remote locations over a network from a user device connected to the network; storing the measurement data at a server connected to the network; comparing the measurement data with one or more pre-determined criteria at the server; contacting a healthcare professional via a device associated with the health care professional connected to the network for review of the measurement data if said one or more predetermined criteria are met; providing the measurement data to the device of the health care professional for review and the healthcare professional subsequently providing a treatment plan based on the measurement data; generating treatment data indicative of the treatment plan and storing the treatment data and providing the treatment data to the user device.

### TECHNICAL FIELD

The present invention generally relates to a method, a system, a computer readable medium of instructions, and/or a computer program product for health assessment and treatment of animals.

### BACKGROUND OF THE INVENTION

Monitoring of animal health in a livestock environment can be difficult due to a number of factors such as the number of livestock, the number of health care professionals which can assess the livestock, and generally the remote location of many of the farms in which the livestock are located.

There exists pig herd recording systems which have functionality to record basic treatment information given to breeding animals (for example Pigtales™ PigWin™, Agrosoft™ WinPig™, Eliteherd™, PigChamp™) etc. However a problem with these systems is that they cannot link identified symptoms (clinical signs of ill health) with certified veterinary-recommended treatment regimes. Also, these systems do not have the functionality to monitor the change in animal welfare status of the animal(s) during a treatment regime. That is, the systems do not calculate dose, track treatments within a treatment regime or distinguish between the different types of treatment that may be given to an animal or a group of animals. These systems also do not notify appropriate personnel where an animal on treatment has, for instance, not been given a welfare assessment or has been given a non-compliant treatment plan.

The treatment information gathered by existing systems is cumbersome to obtain and analyse on an ongoing basis by consulting veterinarians.

Therefore, there exists the need for a method, a system, a computer readable medium of instructions, and/or a computer program product which can alleviate or at least ameliorate one or more of the above problems.

Reference in this specification to any prior publication (or information derived from it), or to any matter which is known, is not, and should not be taken as an acknowledgement or admission or any form of suggestion that the prior publication (or information derived from it) or known matter forms part of the common general knowledge in the field of endeavour to which the specification relates.

### SUMMARY OF THE INVENTION

An aspect of the present invention provides a method for remotely managing the health of animals, the method including: receiving measurement data relating to the health or treatment of one or more animals in one or more remote locations over a network from a user device connected to the network; storing the measurement data at a server connected to the network; comparing the measurement data with one or more pre-determined criteria at the server; contacting a healthcare professional via a device associated with the healthcare professional connected to the network for review of the measurement data if said one or more predetermined criteria are met; providing the measurement data to the device of the healthcare professional for review and the healthcare professional subsequently providing a treatment plan based on the measurement data; generating treatment data indicative of the treatment plan and storing the treatment data at the server; and providing the treatment data over the network to the user device

Advantageously, embodiments of the present invention enhance communication of animal health, including communicating production traits, physiological measures, morbidity and mortality and treatment information between users (such as stockpersons, their supervisors, owners) and the consulting healthcare professional (e.g. veterinarian) without the need for the physical presence of the healthcare professional. Furthermore, the embodiments ideally allow recordal of treatment data (such as indications for treatment, treatments given and welfare measures) in a manner that is convenient for the user and in compliance with regulatory bodies.

It will be appreciated by those persons skilled in the art that heath of an animal includes its welfare, food safety, management husbandry etc.

In a preferred form, the method also includes providing the measurement data to the device of the health care professional over the network for immediate review by the healthcare professional in real time. Providing data in real time ideally allows critical measurement data to be acted upon quickly. For example, non-compliant treatments may be quickly identified, as well as compromised or potentially compromised animals.

As described, the method includes generating treatment data indicative of a treatment plan provided by a healthcare professional based on the measurement data and storing the treatment data at the server. Health care professionals may in advance identify the health events occurring at a location (e.g. farm). The health care professional may design a treatment plan for each significant type of health event identified. This data is entered into the system via healthcare professional terminals that synchronously feed information into the server. Data from the server is synchronised with user devices. Treatment plans may be modified at any time. That is, in an embodiment, the method further includes storing predetermined treatment data indicative of predetermined treatment plans provided by the healthcare professional based on predetermined measurement data. In use, for instance, the method then compares the measurement data with the predetermined measurement data at the server and provides the predetermined treatment data over the network to the user device if the measurement data corresponds to the predetermined measurement data. Also, the method may include providing the measurement data to the device of the healthcare professional for review if the measurement data does not correspond to the predetermined measurement data, and the healthcare professional subsequently providing a modified treatment plan based on the measurement data.

A treatment plan may include, for instance, administering medication(s) individually to a single animal or to a population of animals, or group administered treatments (e.g. via water, feed or environment). These treatments may be administered to clinically affected animals or to populations of animals with the intent of preventing and/or controlling the occurrence of clinical events

In an example, treatment plans may be automatically generated from a veterinary health plan once the symptom and animal class are provided, The user can also collect and send additional measurement data (e.g. pictures and videos, body temperature, body condition change, animal demeanour changes, appetite changes) to the healthcare professional. From this data the user can instigate additional healthcare advice.

In cases where a symptom has not been previously identified, the stockperson may specify the symptom as “unknown”. The healthcare professional is notified of this event so that the event can be investigated and a new treatment plan designed and delivered to the user.

Preferably, the method further includes receiving further measurement data from the user device relating to the health or treatment of said one or more animals following treatment according to the treatment plan, comparing the further measurement data with the treatment data at the server, and providing the further measurement data and the treatment data to the device of the health care professional over the network for review by the healthcare professional based on a result of the comparison. Thus, advantageously, the healthcare professional may track the progress of a treatment and provide further treatment, or end treatment, depending on the further measurement data.

The above pre-determined criteria may create timely notifications to the healthcare professional. Examples of pre-determined criteria that results in a notification being generated include: the number deaths within a class of animal or symptom exceeding a threshold; the number of treatments within a class or symptom exceeding a threshold; animals determined to be severely compromised as assessed by measurement data (e.g. unable to rise, on-going poor appetite or not drinking, body score condition of two or less); a poor response to a treatment plan; inappropriate medication use as measured by medication product, dose, and frequency; or the occurrence of an unknown health event.

Preferably, the measurement data relating to the health or treatment of one or more animals includes pictures, video or text data or identification data, while the measurement data relating to the health or treatment of one or more animals may include the type of animal, the class of animal, the date and time, and the location or number of animals.

In a preferred form, the measurement data relating to the health or treatment of one or more animals includes a unique identifier associated with each animal. Furthermore, the treatment data may include prescribing a drug, indications for treatment, a clinical response to treatment, or a withholding period. For example, the unique identifier is internal to the system, and can be linked to either a unique or non-unique animal identifier. This characteristic allows a stock person to identify animals with a non-unique descriptor such as a “green stripe” or with a unique identifier, such as a numbered ear tag. The system allows for both of these types of animal identification to be recorded for the same animal for the same treatment. Also, there is a unique identifier for a group of animals denoted as “batch identification”. An individual animal can be allocated to the unique batch identifier

In another aspect, the present invention provides a system for remotely managing the health of animals, the system including: a server for receiving and storing measurement data relating to the health or treatment of one or more animals in one or more remote locations over a network connected thereto from a user device connected to the network, wherein the server is arranged to: compare the measurement data with one or more pre-determined criteria and contact a healthcare professional via a device associated with the healthcare professional connected to the network for review of the measurement data if said one or more predetermined criteria are met; provide the measurement data to the device of the healthcare professional for review and the healthcare professional subsequently providing a treatment plan based on the measurement data; generate treatment data indicative of the treatment plan and to store the treatment data at the server; and provide the treatment data over the network to the user device.

Advantageously, the system provides real time information to allow the healthcare professional to assess the health of the animal, including providing visual evidence of any signs or symptoms of ill health. In this embodiment, the server of the system is further arranged to provide the measurement data to the device of the healthcare professional over the network for immediate review by a healthcare professional in real time.

Embodiments of the present invention also ideally allow the healthcare professional to make a diagnosis of a disease endemic to that particular herd. The healthcare professional can then prescribe the correct medication, dose, duration and method of administration based on the measurement data provided by the user. The system also ensures that the medication is being administered by a competent properly trained user (given that the user that is using the system must be trained before having an account).

The system advantageously allows the healthcare professional and the user to monitor the course of treatment, to ensure effectiveness and to ensure that the animals' health is improving. In one form, the user of the system and the healthcare professional may monitor withholding periods and foreign body status associated with an animal or group of animals) including tracking the animal's (group's) movements.

The system also ideally allows the healthcare professional and the user to provide themselves with an overview of the overall health of the animals at any moment in time.

The system advantageously allows the stock person to record health conditions (which do not require treatment), culls, deaths and euthanasia of animals that are not currently on treatment which allows the healthcare professional and the user to monitor these events in the animal population, including the ability of sending notifications when trigger levels are reached so timely health investigations and interventions can be implemented.

In one form, the system automatically produces scheduled reports such as: treated animals that have not been finalised, response to treatment, treatment rates by class/age, potentially compromised animals and medication inventory determinations. In another form, the system produces medication inventory reconciliation estimations based on stock inventory reads, recorded usage and sales history. In another form, the system advantageously allows third parties to receive notification of welfare assessment information to assist them to prepare to receive animals that may require special care on arrival at their destination (e.g., emailing “fitness to load” assessments to receiving stations at an abattoir). In another form, the system advantageously allows for producers to electronically complete and send a National Vendor Declaration for a batch of animals to appropriate third parties.

In an embodiment, the server of the system is arranged to receive further measurement data from the user device relating to the health or treatment of said one or more animals following treatment according to the treatment plan, compare the further measurement data with the treatment data at the server, and provide the further measurement data and the treatment data to the device of the health care professional over the network for review by the healthcare professional based on a result of the comparison.

In another embodiment, the server is further arranged to store predetermined treatment data indicative of predetermined treatment plans provided by the healthcare professional based on predetermined measurement data. The server then compares the measurement data with the predetermined measurement data at the server and provides the predetermined treatment data over the network to the user device if the measurement data corresponds to the predetermined measurement data. Also, the server is further arranged to provide the measurement data to the device of the healthcare professional for review if the measurement data does not correspond to the predetermined measurement data, and the healthcare professional subsequently provides a modified treatment plan based on the measurement data.

In another aspect, the present invention provides a method for remotely managing the health of animals, the method including: receiving measurement data relating to the health or treatment of one or more animals in one or more remote locations over a network from a user device connected to the network, wherein the user device includes a memory having predetermined treatment plans associated with predetermined measurement data; storing the measurement data at a server connected to the network; the user device comparing the measurement data with the predetermined measurement data and providing treatment data indicative of a predetermined treatment plan associated with the predetermined measurement data; the server comparing the measurement data with one or more pre-determined criteria; contacting a healthcare professional via a device associated with the healthcare professional connected to the network for review of the measurement data if said one or more predetermined criteria are met; providing the measurement data to the device of the healthcare professional for review and the healthcare professional subsequently providing a modified treatment plan of the predetermined treatment plan based on the measurement data; generating modified treatment data indicative of the modified treatment plan and storing the modified treatment data at the server; and providing the modified treatment data over the network to the user device.

In another aspect, the present invention provides a system for remotely managing the health of animals, the system including: a server for receiving and storing measurement data relating to the health or treatment of one or more animals in one or more remote locations over a network connected thereto from a user device connected to the network, wherein the user device includes a memory having predetermined treatment plans associated with predetermined measurement data, and the user device is arranged to compare the measurement data with the predetermined measurement data and provide treatment data indicative of a predetermined treatment plan associated with the predetermined measurement data, and wherein the server is arranged to: compare the measurement data with one or more pre-determined criteria and contact a healthcare professional via a device associated with the healthcare professional connected to the network for review of the measurement data if said one or more predetermined criteria are met; provide the measurement data to the device of the healthcare professional for review and the healthcare professional subsequently providing a modified treatment plan of the predetermined treatment plan based on the measurement data; generate modified treatment data indicative of the modified treatment plan and to store the modified treatment data at the server; and provide the modified treatment data over the network to the user device.

### BRIEF DESCRIPTION OF DRAWINGS

Embodiments of the present invention will now be described in further detail with reference to the accompanying drawings. It is to be understood that the particularity of the drawings does not supersede the generality of the preceding description of the invention.

FIG. 1 is a schematic diagram showing a system and method for health assessment of animals according to an embodiment of the invention;

FIG. 2a is a flow diagram illustrating an example method of the invention;

FIG. 2b is a detailed schematic diagram showing an example system and method for health assessment of animals according to an embodiment of the invention;

FIG. 2c is a block diagram illustrating the data structure associated with the database of an embodiment of the system and method of the present invention;

FIG. 2d is an object diagram showing the database of an embodiment the present invention in operation;

FIG. 3a is a flow diagram with corresponding example screen shots for beginning a new individual treatment according to an embodiment of the invention;

FIG. 3b is a flow diagram with corresponding example screen shots for beginning a new routine treatment according to an embodiment of the invention;

FIG. 3c is a flow diagram with corresponding example screen shots for beginning a new group treatment according to an embodiment of the invention;

FIG. 4 is a flow diagram for notifying a healthcare professional according to an embodiment of the invention;

FIG. 5 is a flow diagram with corresponding example screen shot of alerting a user of required actions to be carried out according to an embodiment of the invention;

FIG. 6 is a flow diagram with corresponding example screen shot for alerting a user regarding the withhold status of an animal (or a group of animals) stock according to an embodiment of the invention;

FIG. 7a is an example screen shot illustrating the selection of one or more health options available to the user from the home screen;

FIG. 7b is an example screen shot illustrating the selection of one or more health options available to the user from a menu from the home screen and the options available after selecting one of the home screen buttons;

FIG. 8 is an example screen shot illustrating the selection of one or more options relating to the animal type;

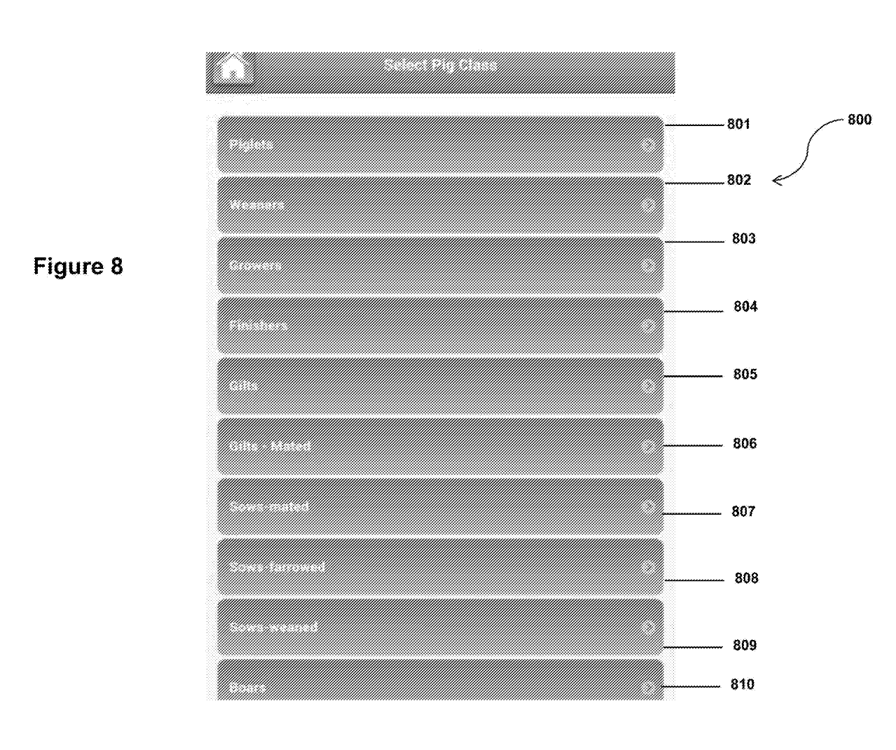

FIG. 9 is an example screen shot illustrating a list of one or more reasons that have been identified by a healthcare professional;

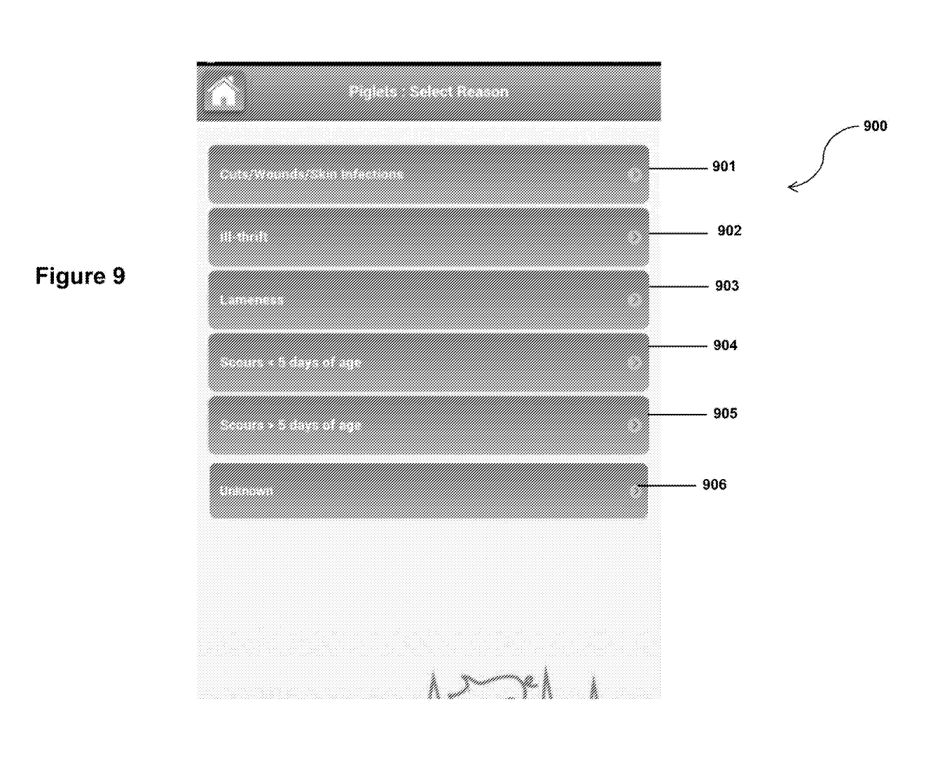

FIG. 10 is an example screen shot illustrating the entering detail of the animal(s) to be treated;

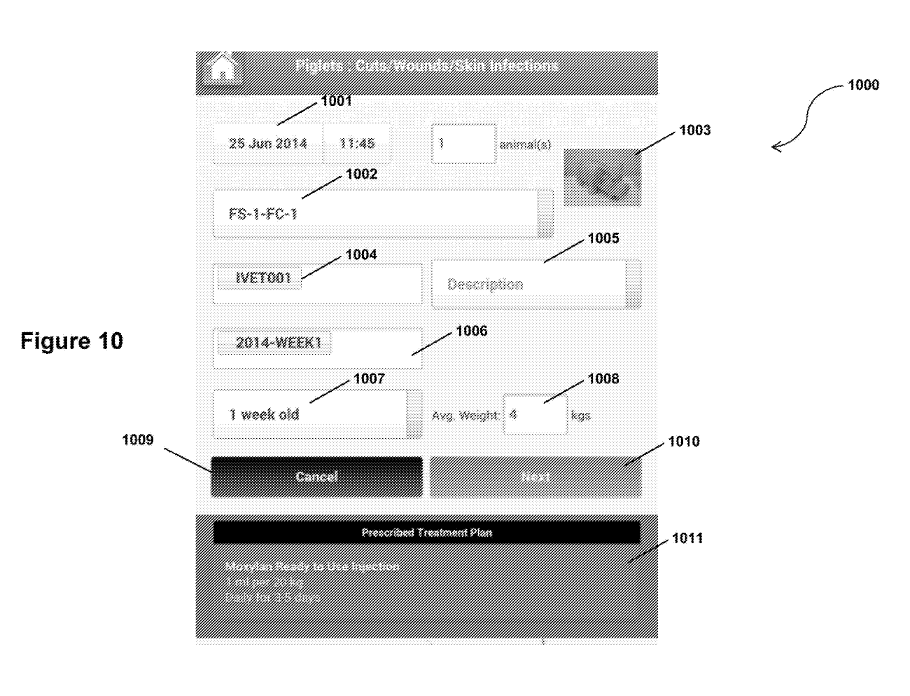

FIG. 11 is an example screen shot illustrating the capture of digital data via photo or video;

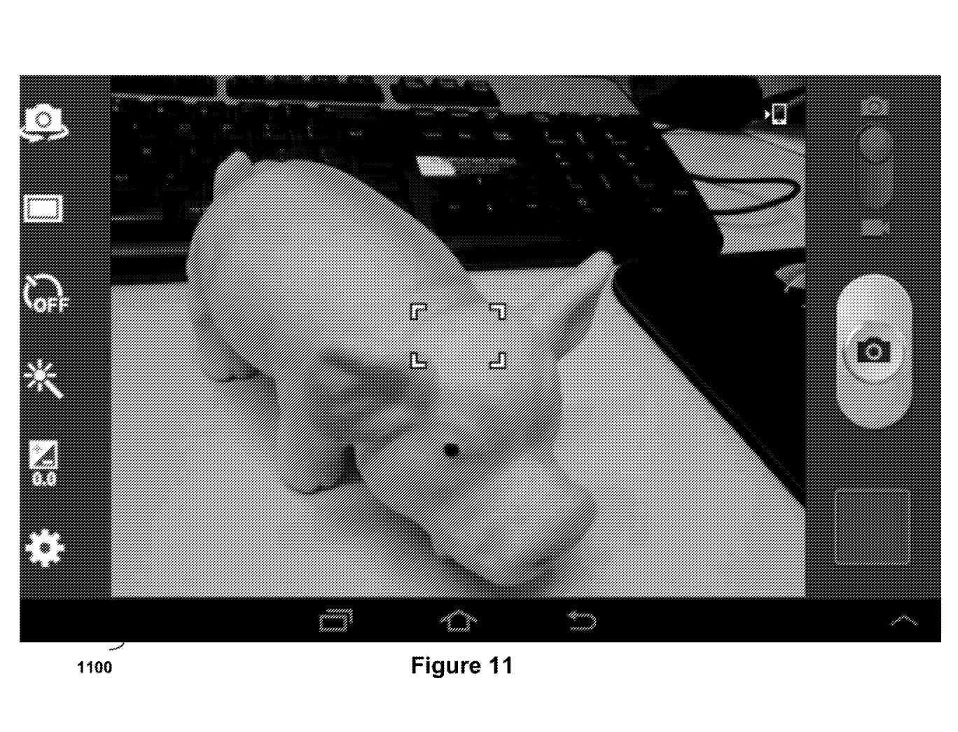

FIG. 12 is an example screen shot illustrating entering detail of the health assessment of the animal(s);

FIG. 13 is an example screen shot illustrating identification of the user capturing the measurement data relating to the animal(s);

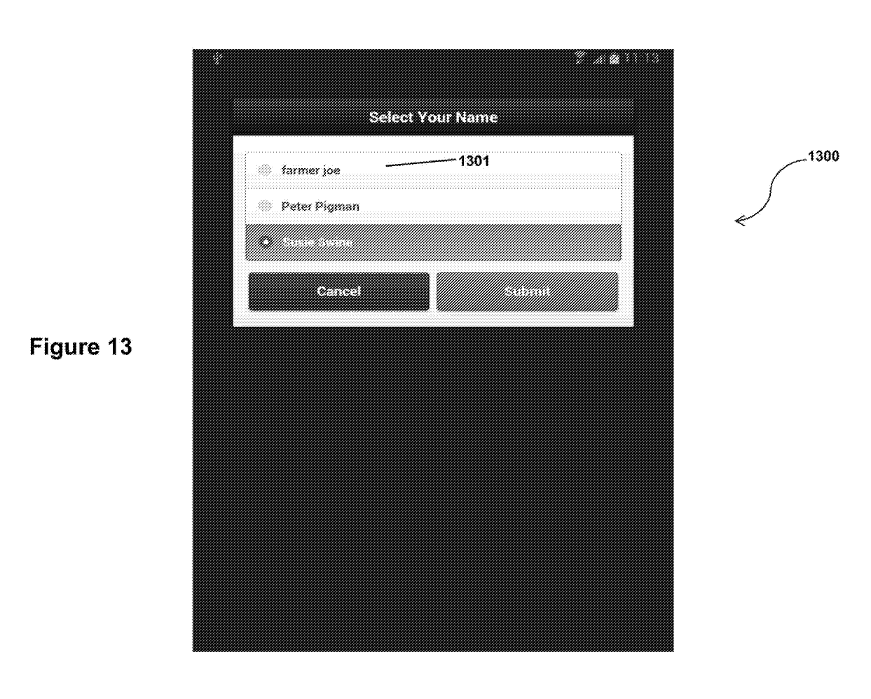

FIG. 14a is an example screen shot from the user device illustrating the prescribed individually administered medication regime for the animal(s);

FIG. 14b is an example screen shot illustrating the capture of data relating to a foreign body occurrence (e.g. retained or lost needle);

FIG. 14c is an example screen shot illustrating the capture of data required if a prescribe medication is not administered as prescribed;

FIG. 15a is an example screen shot from the user device illustrating an overview of the health assessment and treatment history (denoted the Treatment Card) for the animal(s) individual treatment regime;

FIG. 15b is an example screen shot illustrating menu options available from the Individual Treatment Card;

FIG. 16 is an example screen shot illustrating a list of the animals currently under treatment grouped by class of animal;

FIG. 17 is an example screen shot illustrating a list of the animals currently requiring attention grouped by class of animal;

FIG. 18 is an example screen shot of entering health assessment data for the animal(s);

FIG. 19 is an example screen shot illustrating data entry required from the user when “End Treatment” button is selected;

FIG. 20 is an example screen shot on the user device after the “Start Routine Treatment” button is pressed;

FIG. 21 is an example screen shot illustrating the medication details entered for a Routine Treatment (medication and dose which are populated from Treatment Plan database);

FIG. 22 is an example screen shot illustrating the next Routine Treatment medication due for a Batch of animals generated from the pre-scheduled Routine Treatment Plan;

FIG. 23a is an example screen shot illustrating the Routine Treatment Card for a batch of animals;

FIG. 23b is an example screen shot of the menu options available from the Routine Treatment Card;

FIG. 24 is an example screen shot from the user device illustrating the Types of Group Treatments Plans;

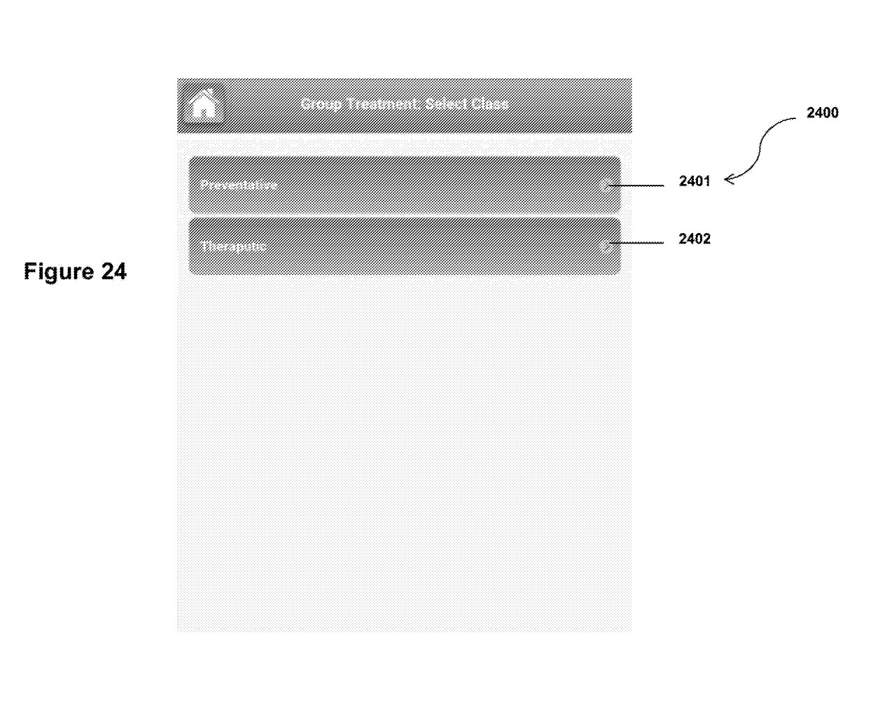

FIG. 25 is an example screen shot illustrating Group Treatment Plan available within a Group Treatment Plan Type;

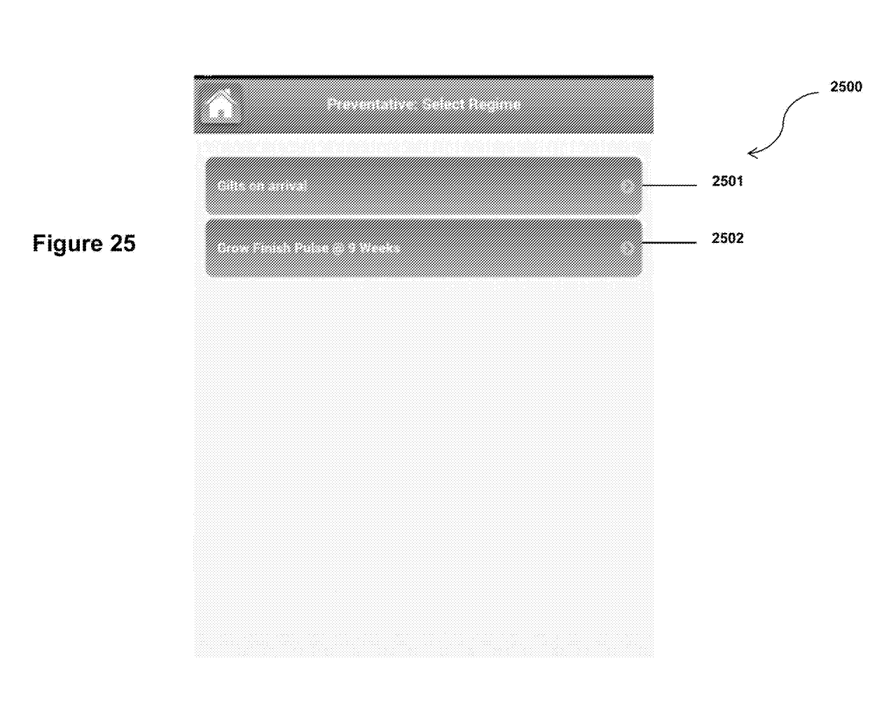

FIG. 26 is an example screen shot illustrating the animal group details entered for a Group Treatment;

FIG. 27 is an example screen shot illustrating the medication details entered for a Group Treatment (along with the instructions, medication and dose which are populated from Treatment Plan database);

FIG. 28 is an example screen shot illustrating the Group Treatment Card for a batch of animals;

FIG. 29 is an example screen shot illustrating the animals currently on withhold from sale and animals that have a foreign body status (egg retained needles);

FIG. 30a is example screen shot illustrating the list of treated animals assigned “to be culled”;

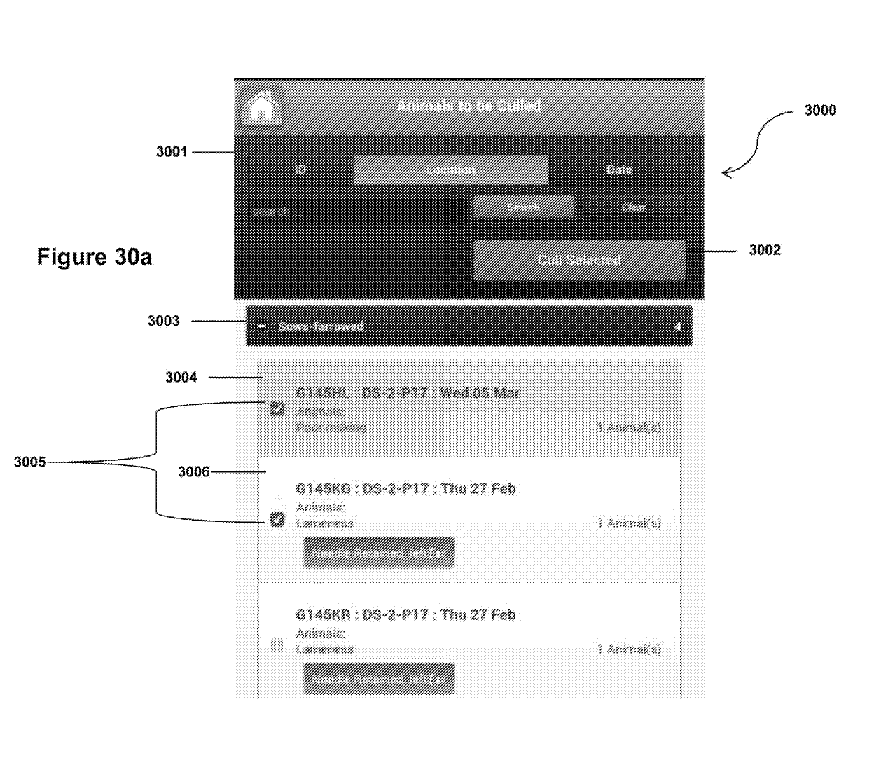

FIG. 30b is an example screen shot illustrating the confirmation of animals culled;

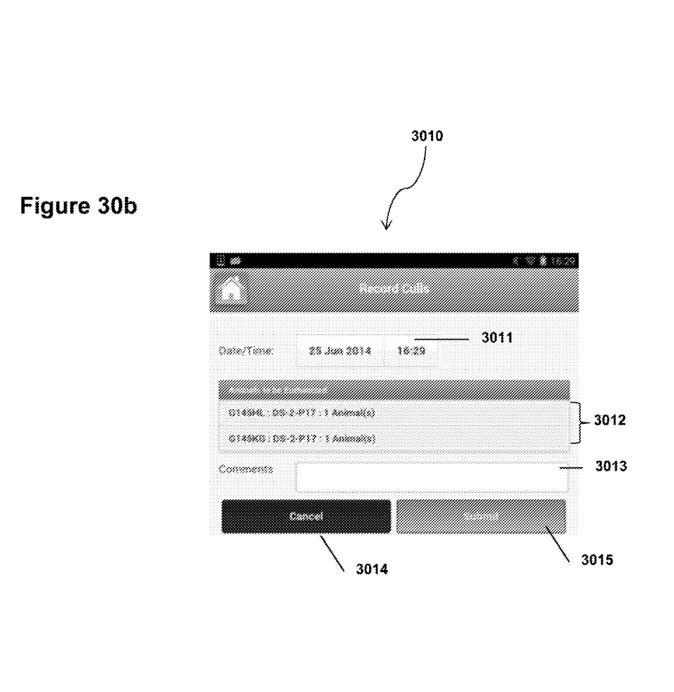

FIG. 31a is example screen shots illustrating the list of treated animals assigned “to be euthanized”;

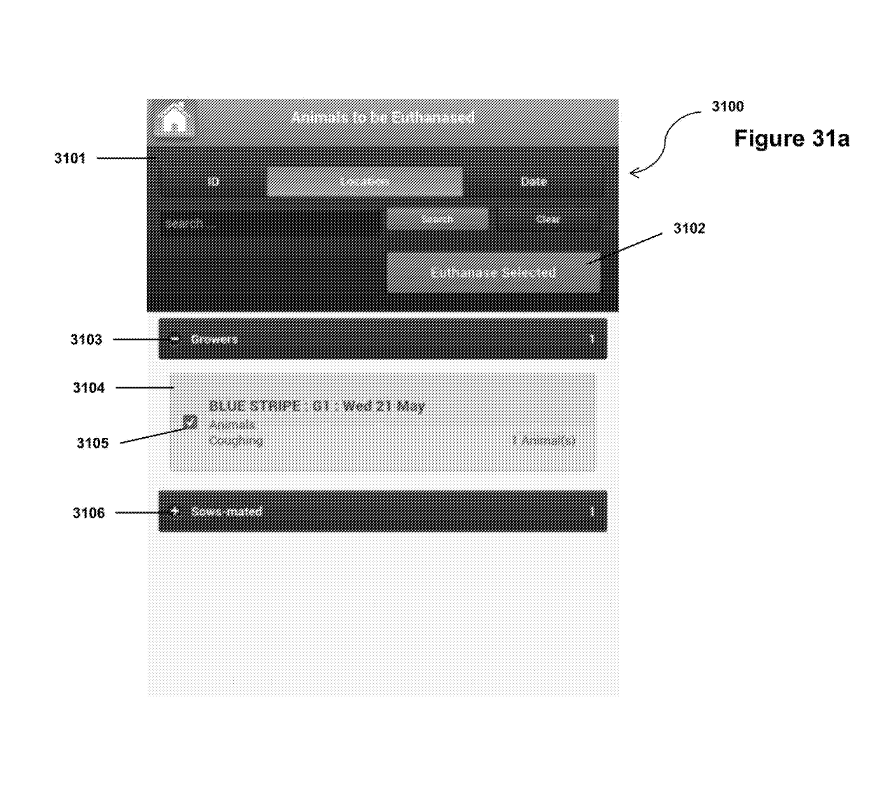

FIG. 31b is an example screen shot illustrating the confirmation of animals euthanized;

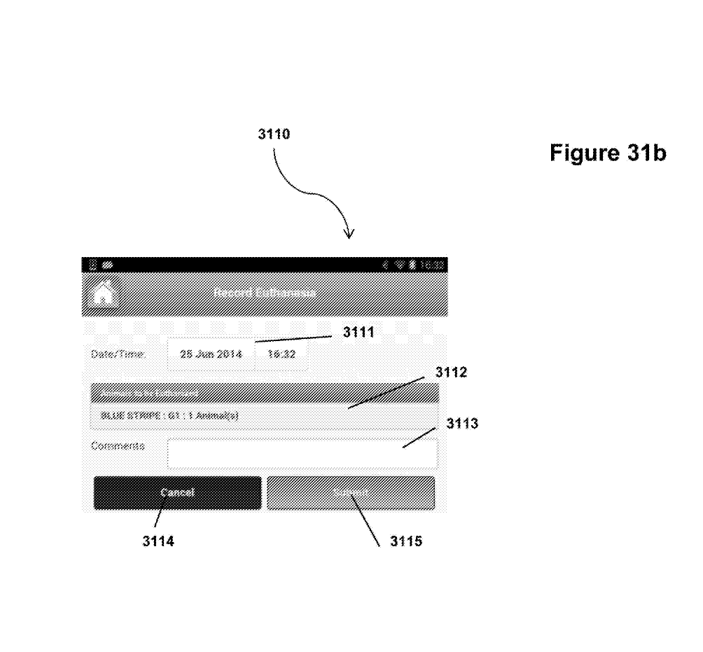

FIG. 32 is an example screen shot of the screen when the “Animal Removals” button is pressed followed by the “Found Dead” button;

FIG. 33 is an example screen shot of the “Recent Deaths” list;

FIG. 34 is an example screen shot illustrating the entire treatment history for a Batch of animals including Individual, Routine and Group treatments;

FIG. 35 is an example screen shot illustrating the management of User Permissions;

FIG. 36 is an example screen shot of a “Dashboard” displaying current treatment, welfare and animal removal information displayed for the entire animal population and for each class of animal;

FIG. 37 is an example screen shot of historical Individual Treatments (on an individual animal basis) displayed for each animal class-reason combination for contiguous time periods and for the corresponding period the previous year;

FIG. 38 is an example screen shot that lists the details of Individual Treatments administered, including their compliance status with respect to medication use. This report can similarly be generated for Routine and Group Treatments;

FIG. 39 is an example screen shot that lists the response to treatment from Individual Treatments administered;

FIG. 40 is an example screen shot illustrating the history for a particular individual treatment case;

FIG. 41 is an example screen shot reporting of the cross-tabulation between health assessment and response to treatment for a class of animal-reason combination;

FIG. 42 is an example screen shot of a report presenting the treatments administered data in a form that complies with the requirements of a quality assurance program;

FIG. 43 is an example of a traditional reporting method relating to treatment records used in the pig industry;

FIG. 44a is an example screen shot of an notification email sent to a healthcare professional when there has been a non-compliance in treatment administration;

FIG. 44b is an example screen shot from the web-based interface of an exception listing report of treatment information;

FIG. 45 is an example screen shot of the review history made by the healthcare professional pertaining to exception reported data;

FIG. 46a is an example screen shot displaying medications, their instructions, reasons and animal classes;

FIG. 46b is an example screen shot displaying the user interface for allocating and prioritising Individual Treatments (e.g. medications) along with accompanying instructions to treat health ailments within an animal class by a healthcare professional;

FIG. 46c is an example screen shot displaying the user interface for entering prescribed individual treatment information by the healthcare professional;

FIG. 47 is an example screen shot displaying the user interface for allocating Routine (also known as Fixed Dose) Treatments along with accompanying instructions to prevent, control and/or treat health conditions within a group of animals by a healthcare professional;

FIG. 48 is an example screen shot displaying the user interface for allocating Group (also known as Water Dose) Treatments along with accompanying instructions to prevent, control and/or treat health conditions within a group of animals by a healthcare professional;

FIG. 49a is an example screen shot displaying the user interface for selecting and sending treatment information to mobile devices;

FIG. 49b is an example screen shot displaying Routine and Group Treatments as a pre-scheduled program for a batch of animals;

FIG. 50 is a further exemplary flow chart of a method of the invention in use;

FIG. 51 shows a treatment plan according to an embodiment of the invention.

### DETAILED DESCRIPTION

The following description describes an embodiment of the system in the context of a number of devices connected to the Internet and operating via a website and server. It should be appreciated that the system could operate on a 3G or more advanced mobile telephone network or the like.

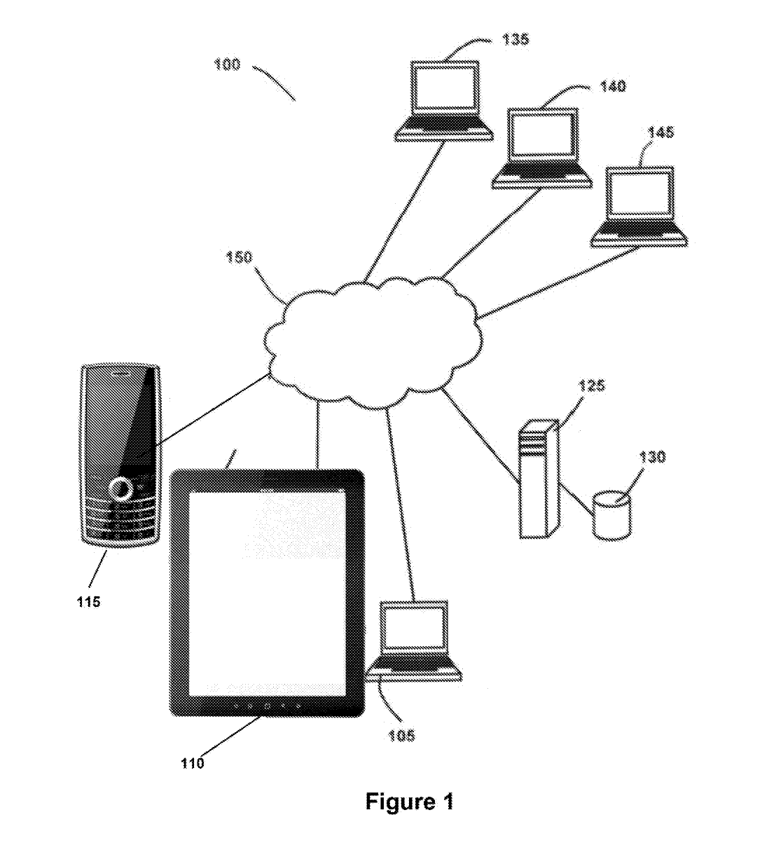

Referring to FIG. 1 there is shown an example system 100 for the health assessment and treatment of animals. The system 100 includes one or more user devices 105, 110, 115 (which may include PC's, mobile communication devices or tablet computers), one or more healthcare professional devices (i.e. terminals) 135, 140, 145 each of which is associated with a healthcare professional such as a veterinarian. A database 130 is in communication with a server 125 and the user devices 105, 110, 115 and the healthcare professional terminals 135, 140 and 145.

In operation, the user devices 105, 110, 115 which may be in remote locations relative to each other, elect to provide measurement data in relation to one or more animals, and the healthcare professionals 135, 140, 145, log on or connect to the server 125 via a network 150 such as the Internet or a mobile telephone network and. The measurement data from the user device 105, 110, 115 may have been recorded at a time where the device was not connected to the network 150. In such a case the information is stored at least until all measurement data can be provided to the server 125 and stored in the database 130.

Upon receipt of the measurement data at the server 125, the healthcare professionals via terminals 135, 140 or 145 may log on or connect to the server to obtain the measurement data uploaded by the user associated with terminals 105, 110 and 115. Advantageously, the system and method proactively alerts a healthcare professional associated with terminals 135, 140 or 145 if it is found via the measurement data that one or more predetermined criteria have been met. These predetermined criteria, may include for example a case of unknown aetiology, and/or of urgent assessment in which the mortality rate reported in the measurement data is higher than a predetermined threshold or, if in the course of one or more sets of measurement data the health of the animal has not recovered as expected despite treatment and dosage of medication.

The measurement data may also reside on the server 125 until such time as the healthcare professional associated with terminal 135, 140 and 145 accesses the server and reviews the measurement data to provide a response to the measurement data in the form of treatment data.

The treatment data may include prescribing a medication, mode of administration, dose, frequency of administration, indications for treatment, clinical response to treatment or a withholding period.

The treatment data is uploaded to the server 125 via network 150 and this data is disseminated to the appropriate user device 105, 110, 115 for the user to carry out the instructions of the healthcare professional and, for example, administer medication or take other action.

It will be appreciated that the above mentioned method and system may be an iterative one in that measurement data may be obtained and treatment data may be provided and further measurement data may be provided so as to provide further treatment data and/or to end treatment.

Each of user devices 105, 110 and 115 preferably include audio/visual inputs in which to take photographs or video of the animals, or include other digital data associated with the heath of the animal and/or its environment (such as body temperature, respiratory rate, heart rate, feed and water consumption patterns, air quality measures, GPS, etc.). This advantageously assists the healthcare professional in making an assessment and providing treatment data.

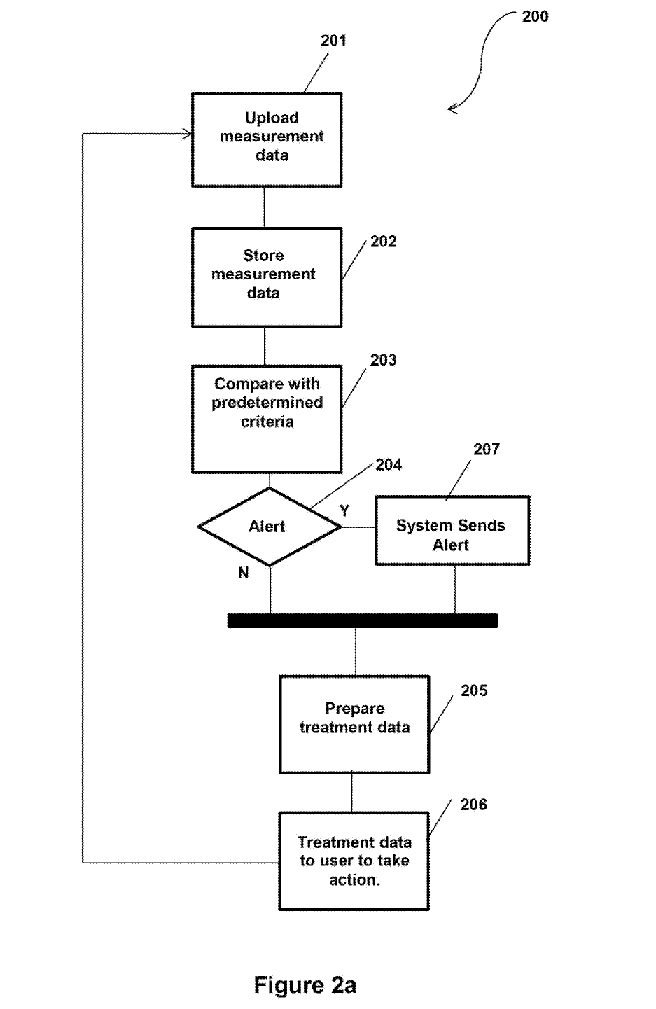

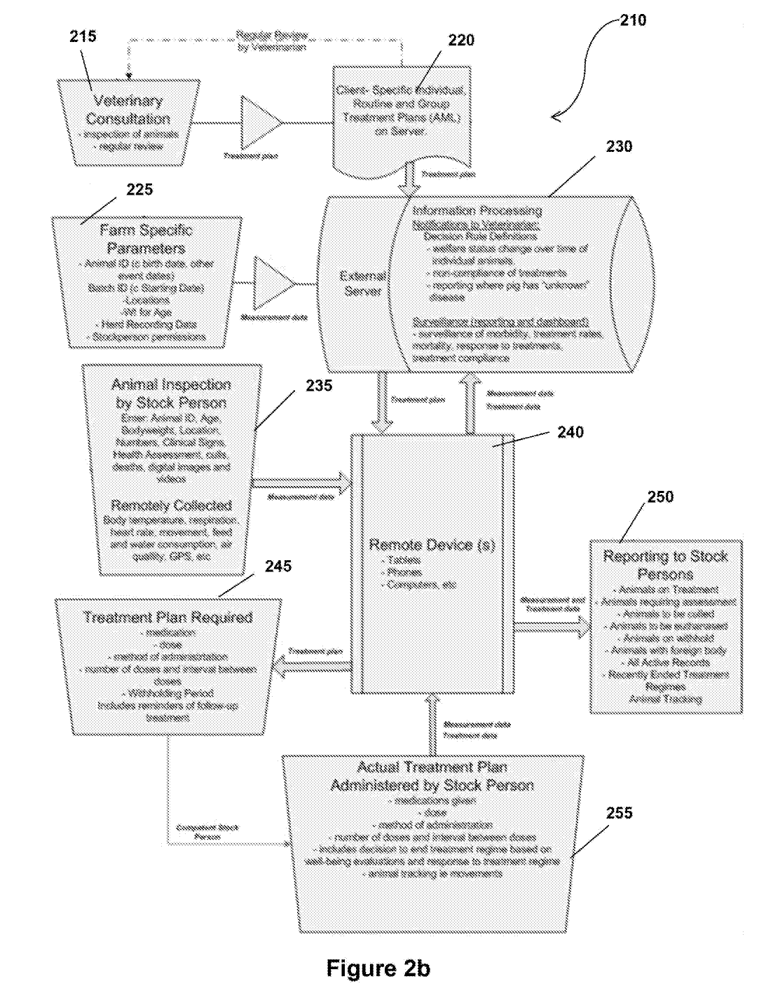

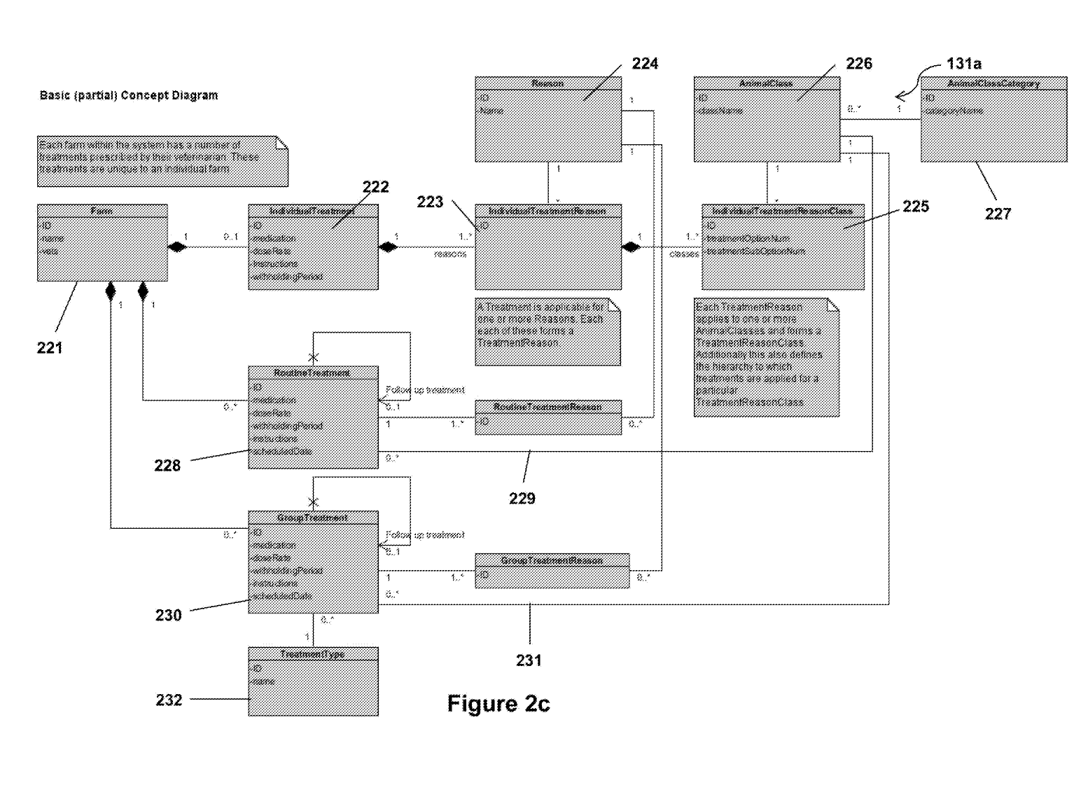

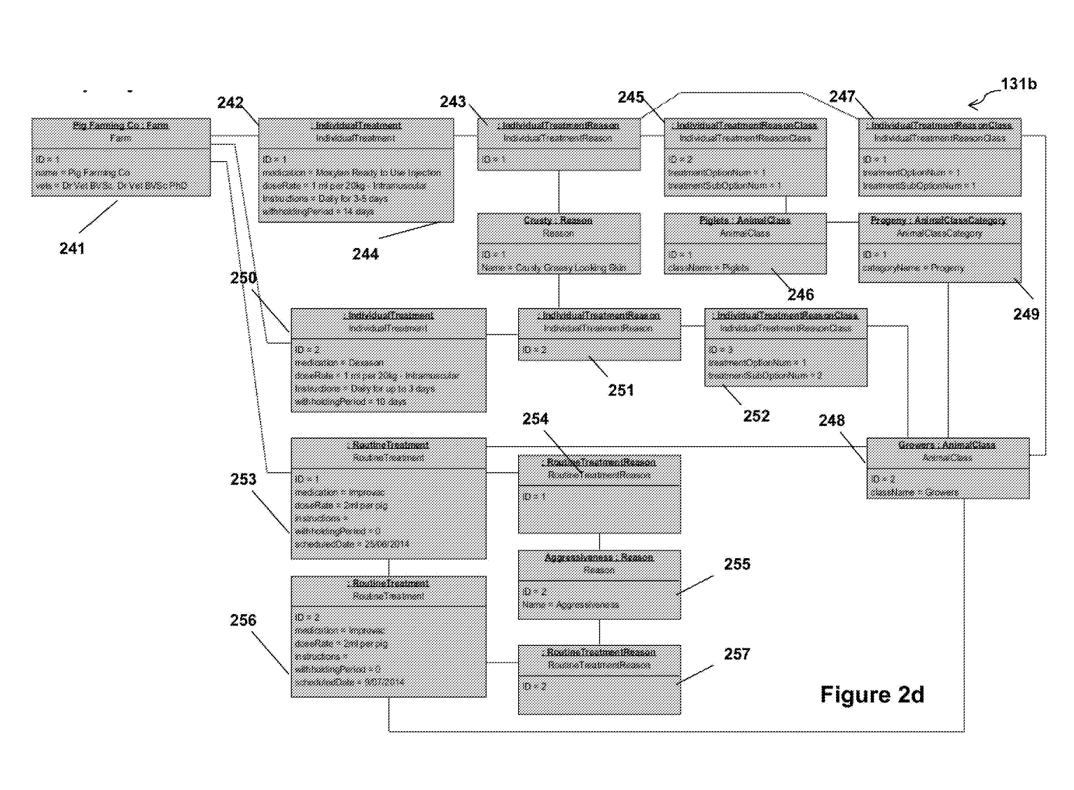

FIG. 2a is a flow diagram illustrating a preferred embodiment of the invention. The method 200 is initiated by a user associated with user device 105, 110 and 115 where at step 201 the user uploads measurement data in relation to the health of one or more animals at a remote location. The measurement data may take the form of the type of animal, the class of animal, typical symptoms, date and time, location, number of animals effected and a unique identifier associated with each animal, animal batch, the age of the animal, the weight of the animal, an overall health score of the animal, whether the animal is eating or not, whether the animal is drinking or not, an overall body score and any other data and comments together with photographs or video.

Control then moves to step 202 in which the measurement data is uploaded from the user associated with user device 105, 110, 115 and stored on the database 130 of server 125. The measurement data may be uploaded by a user device 105 in the form of a mobile communication device over a telecommunications network or maybe a tablet 110 connected to the internet or a computer 115 also connected to the internet or may be via 3G or 4G or satellite data communication or the like.

At step 202 the measurement data is stored at the server 125 for later review by one or more healthcare professionals who are associated with terminals 135, 140 and 145. Control then moves to step 230 in which the measurement data on the server 125 is compared with one or more predetermined criteria on the server 125. The purpose of this step is to automatically determine whether or not, based on measurement data there is a possible emergency situation associated with the healthcare of the animals associated with the measurement data uploaded at step 201.

The predetermined criteria may include: the number of pig deaths within a class or symptom exceeding a threshold; the number of treatments within a class or symptom exceeding a threshold; animals severely compromised as assessed by the users health score (e.g. unable to rise, on-going poor appetite or not drinking, body score condition lower than a predetermined threshold); poor response to initial treatment plan; inappropriate medication use as measured by medication product, dose, and frequency; occurrence of an unknown health event;

For example if the measurement data indicates that the mortality rate on the farm is higher than a predetermined threshold or if an animal has not recovered as expected from a previous treatment or for any other reason dictated by the predetermined criteria, an alert is sent to the healthcare professional. The server 125 selectively initiates immediate contact with the healthcare professional via the healthcare professional terminal 135, 140 or 145.

It will be appreciated that one or more healthcare professionals may be associated with the system and also associated with a particular user. For example one farm may have a handful of healthcare professionals who can if required, attend to the alert. Once the comparison with predetermined criteria occurs at step 230, control moves to step 204 where an alert may be triggered. At step 204, in the event the measurement data does meet one or more of the predetermined criteria, control moves to step 205 in which the healthcare professional associated with terminal 135, 140 or 145 is alerted. The alert may take the form of an email or SMS message or the like to the healthcare professional terminal 135, 140 and 145 or both.

Control then moves to step 240 in which the healthcare professional provides a response to the measurement data by applying their skills and the system generates treatment data. The treatment data may include, for example prescribing a medication, indications for treatment, a clinical response to a treatment or determining a withholding period. Additional treatment data may include the delivery method (e.g. syringe size, needle size and location of administration). Aggregated treatment may be used to determine group treatment advice.

Control moves to step 240 where the treatment data is delivered to the user for action. Control then returns to step 201 in which the process may be repeated. It will be appreciated that the system and method of the present invention may be an iterative process particularly in the case of treatment of animals such that multiple measurement data over time may be required together with treatment data sent by the healthcare professional in order to ensure the health of the animal.

It will be appreciated that at step 207 the system 100 may schedule an alert to the healthcare professional via terminals 135, 140, 145 at a suitable time (such as the next day) or the healthcare professional may simply log into the server 125 via terminals 135, 140, 145 to obtain the relevant measurement data and in their own time review the measurement data and allow the system to generate treatment data based on the measurement data received.

If at step 204 it was determined that there was no urgency in contacting the healthcare professional, the server 125 stores the measurement data for review by one or more healthcare professionals and control moves to step 240 where the treatment data prepared by the healthcare professional is reported to the user via user devices 105, 110 and 115 to take action. Control then returns to step 201 to obtain further measurement data if required in order to provide further treatment data if required.

It will be appreciated that any animal may be treated by the system and method of the present invention but for ease of reference an example will be provided in the case of pigs.

FIG. 2b is a detailed schematic diagram showing the system and method of the present invention and allowing consulting healthcare professionals to implement and monitor the delivery of timely, explicit and specific animal health advice and actions to stock persons using the system for management of animals which have been identified with a health issue. A healthcare professional which may include a veterinarian may inspect 215 the animals or conduct a regular review and provide a treatment plan. The treatment plan is then gathered and forwarded 220 to an external server 230 such as that is described with reference to FIG. 1. The server 230 may receive measurement data from farm specific data 225, such as animal identification lists along with event histories, farm locations, weight for age relationship and stock person permissions. Preferably this information is automatically updated where possible.

The external server component 230 conducts information processing including notifications to the healthcare professional including decision rule definitions, welfare status changes over time of individual animals and non-compliance of treatments. It also conducts surveillance and regular reporting including surveillance of morbidity, treatment rates, mortality, response to treatments and treatment compliance.

The external server 230 is in data communication with remote devices 240 such as tablets, phones, computers and the like such as those as described with reference to FIG. 1 (for example 105, 110 and 115). The remote device 240 receives measurement data from animal inspection by stock person 235 in which the stock person may enter animal identification, age, bodyweight, location, numbers, clinical signs, wellbeing measurements and the like. In addition, there are provisions to accept physiological and environmental measurement data from remote sensing devices, such as body temperature, respiratory rate, heart rate, feed and water consumption patterns, air quality measures, GPS, etc.

The remote devices 240 may communicate with the external server 230. The remote device may also report out via report function 250 displaying animals which are currently on treatment, animals which require assessment, animals which are currently on withhold from sale, animals with foreign bodies, all active records, death lists, to be culled, to be euthanized and recently ended treatment regimes, for example.

The remote device 240 may also provide a treatment plan 245 including medication, dose, method of administration, number of doses and interval between doses and a withholding period. This data may be delivered to a stock person 255 to carry out the instructions including medications given, the dose, the method of administration, the number of doses, commencement date, and interval between the doses, including a decision to end treatment based on wellbeing evaluations and the response to the treatment regime. Data from this component 255 is fed back to the remote device 240 which in turn feeds the data back to the external server 230.

As described, with reference to FIG. 2b, the system 210 provides methods for healthcare professionals to remotely monitor and review the health and treatment effectiveness of animals which may assist in decisions to subsequently deliver and update the treatment plans for a farm. Methods are available for real time monitoring of treatment measurement data. This includes notifications and methods via a web interface.

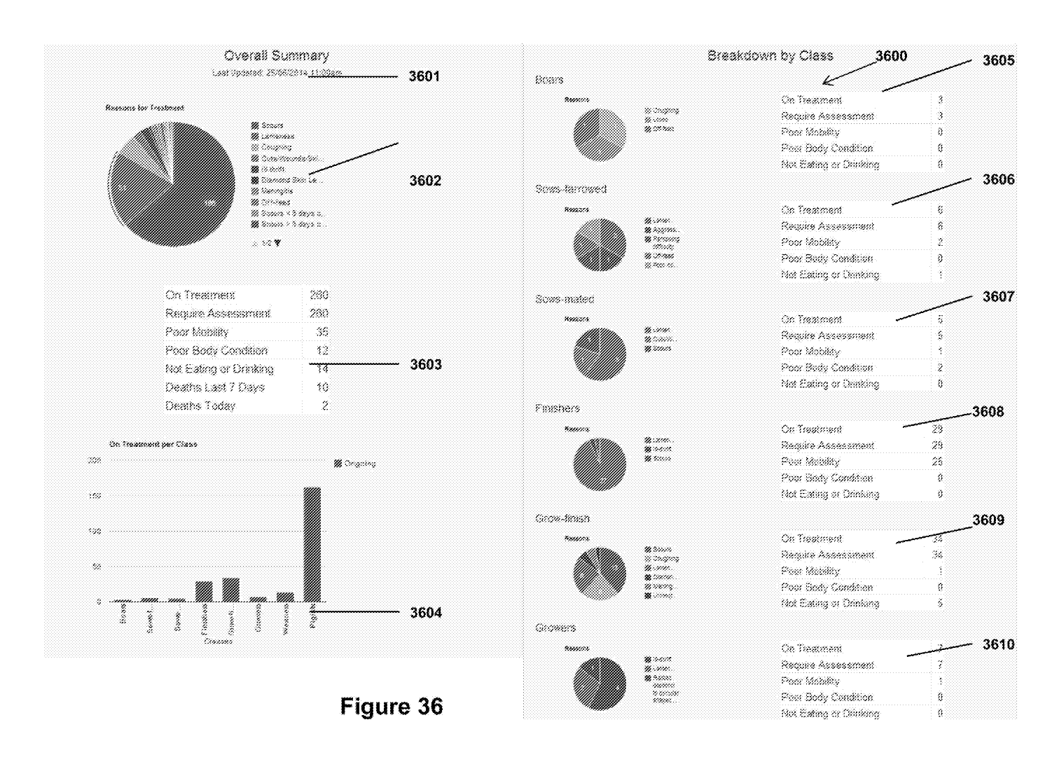

FIG. 36 illustrates a dashboard that shows a summary of the health status of a farm at the present time. It shows when the farms data was last updated 3601, the number and reason for all treatment cases 3602 and a summary of the welfare status (including deaths and euthanasia) of the herd 3603. A summary of treatments per animal class 3604 is shown with a breakdown for these classes also shown 3605-3610.

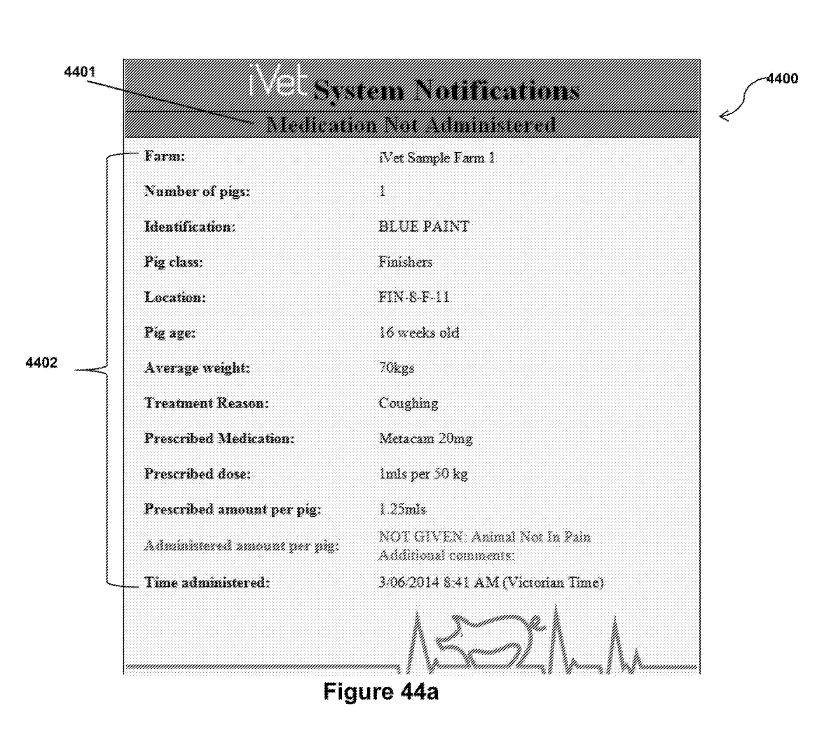

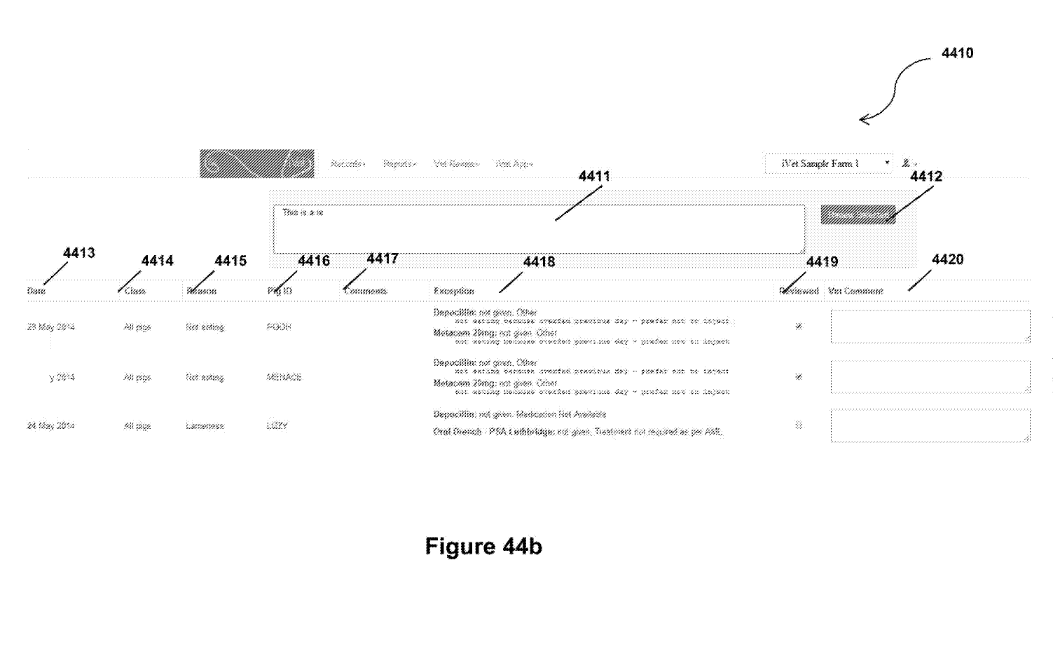

Methods are available for reviewing noteworthy and non-compliant treatment data. FIG. 44b shows a method for reviewing such data and acknowledging that it has been reviewed. Each new noteworthy or non-compliant treatment is displayed 4413-4418, where the healthcare professional is able to acknowledge review 4419 and provide a comment 4420. A healthcare professional can provide an overall comment 4411 before submitting the review 4412.

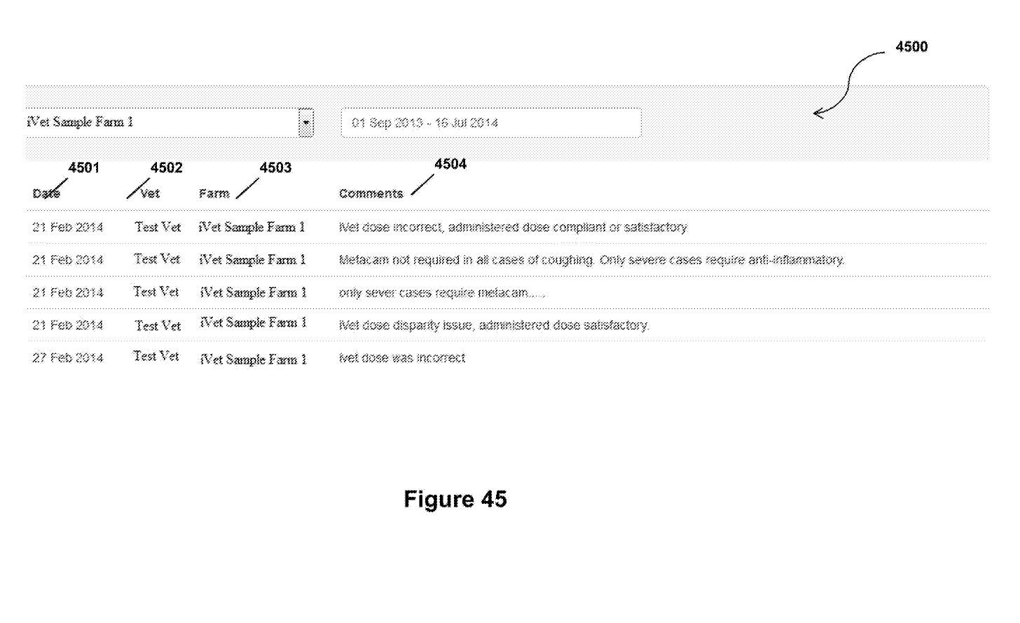

FIG. 45 shows a method for viewing previously reviewed data. Methods are available for analysing treatment measurement data. This is in the form of reports and data exporting.

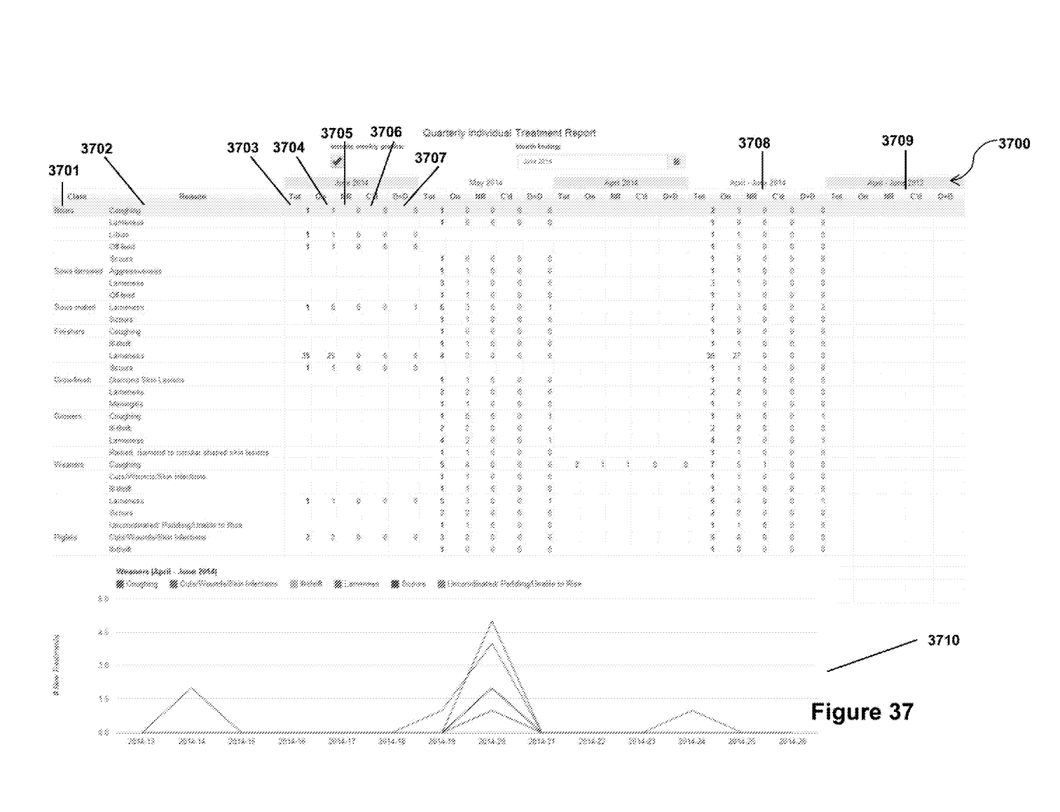

FIG. 37 illustrates a report that shows a summary of treatment effectiveness over a 3 month period. It shows the class 3701, treatment reason 3702 and the total treated 3703, number still on treatment 3704, number requiring a new plan 3705, number culled 3706, number died and destroyed 3707 for each month. A total for the period 3708 and for the same period the previous year 3709 is also shown. This report has provisions for showing a breakdown for each class over the period 3710.

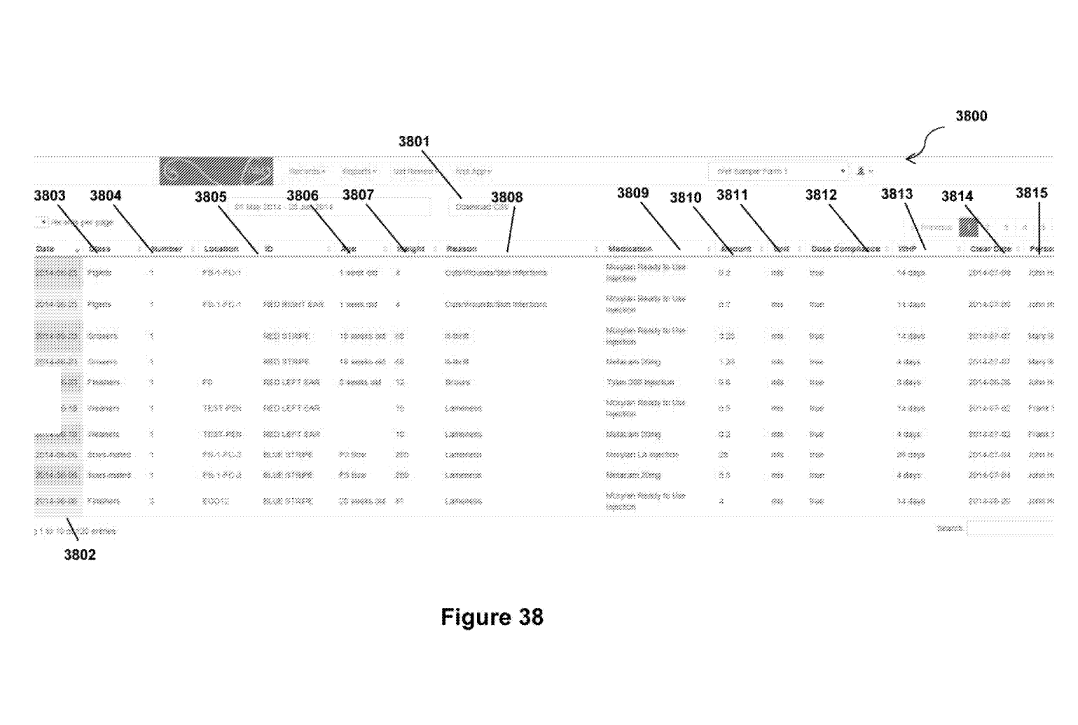

FIG. 38 illustrates a report showing all medication administrations over an adjustable time period. It shows the date 3802, class 3803, number of animals 3804, location, id 3805, age 3806, weight 3807, reason 3808, medication 3809, amount administered 3810, the unit 3811, compliance 3812, WHP 3813, clearance date 3814 and person who administered 3815. This data can be exported to CSV file 3801 for manipulation using external tools (e.g. Excel).

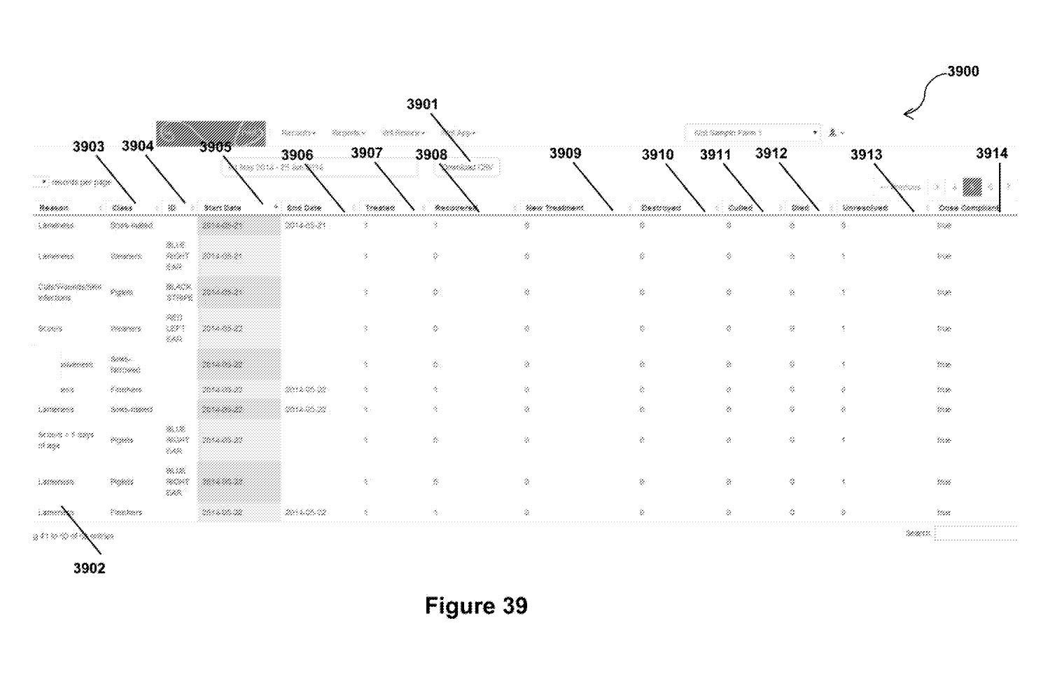

FIG. 39 illustrates a report showing the effectiveness of particular treatment cases over an adjustable time period. It shows the reason 3902, class 3903, id 3904, treatment start date 3905, treatment end date 3906, number treated 3907, number recovered 3908, number requiring new treatment 3909, number destroyed 3910, number culled 3911, number died 3912, number unresolved 3913 and whether all medication administration was compliant 3914. This data can be exported to CSV file 3901 for manipulation using external tools (E.g. Excel).

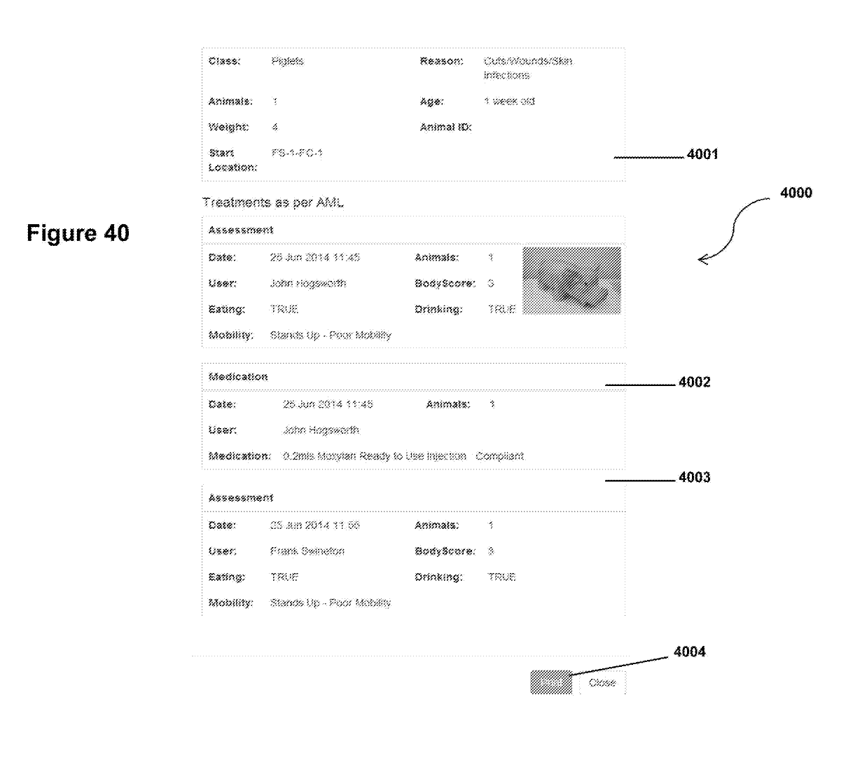

FIG. 40 illustrates a report showing the treatment history for a specific treatment case. It shows the animal details 4001, welfare assessments 4002, medications administered 4003, and other events (animal movements, culls, euthanasia and deaths). This report can be printed if required 4004.

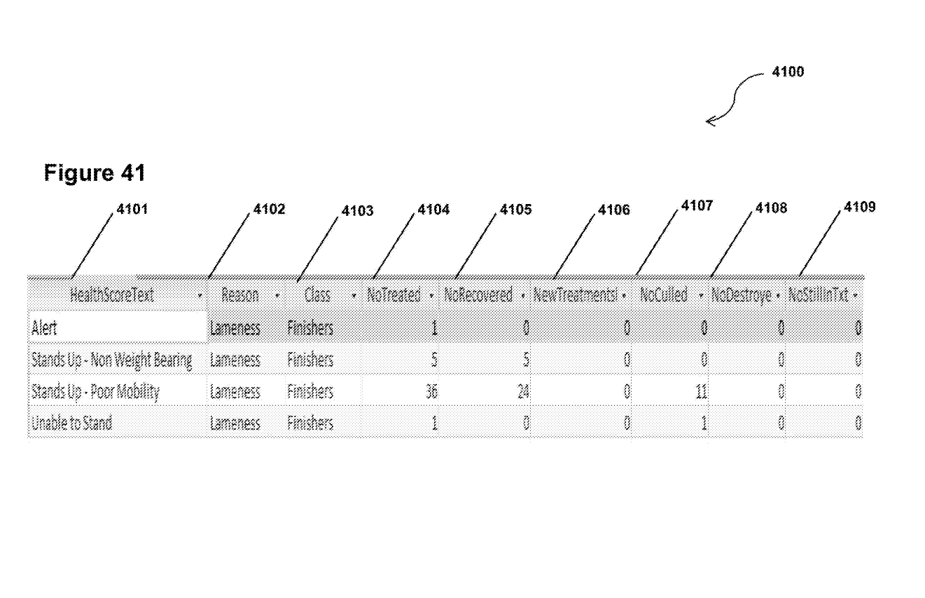

FIG. 41 illustrates a welfare status by treatment response report for a condition in a class of animal. It is produced from the measurement data available, showing the relationship between welfare status at intervention 4101 and the effectiveness of treatment 4104-4109 for a particular reason 4102 and animal class 4103.

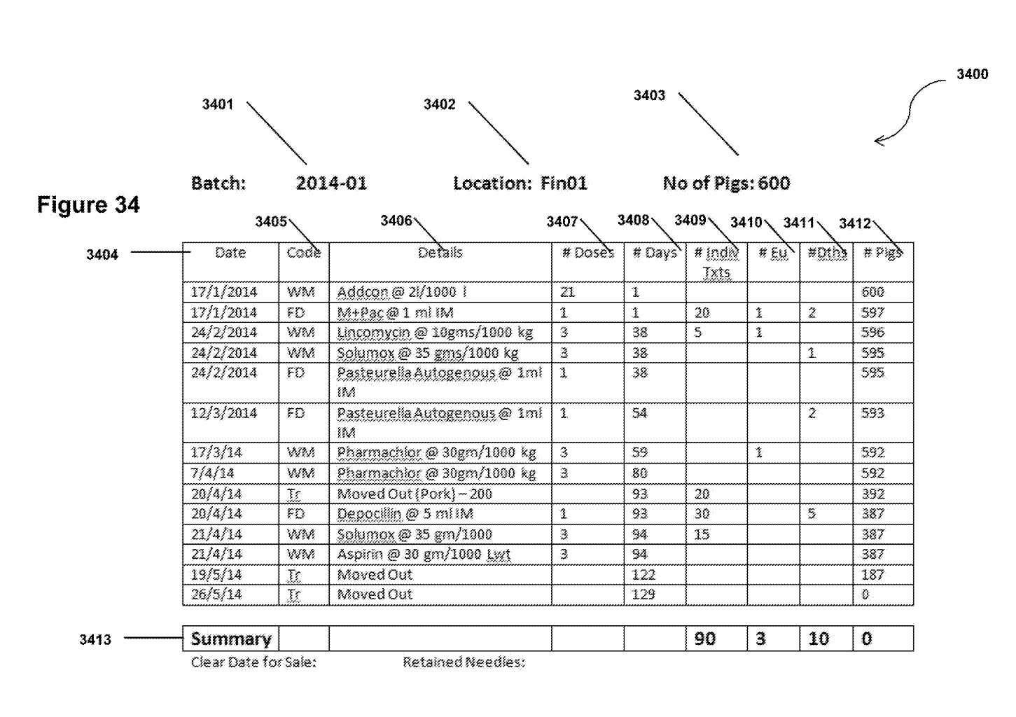

FIG. 34 illustrates an example of a Batch report, which is produced from the measurement data available, showing all the group events for a particular batch of animals. It includes the batch 3401, current location 3402, starting number of animals 3403 and all treatment events. For each event the following is shown: date 3404, code 3405, details 3406, number of doses 3407 (if applicable), days of age at the time 3408, and a count of individual events for animals from the batch between the last group event and this event (Individual Treatments 3409, Euthanasia 3410 and Deaths 3411). The number of animals remaining in the group after the event is also shown 3412.

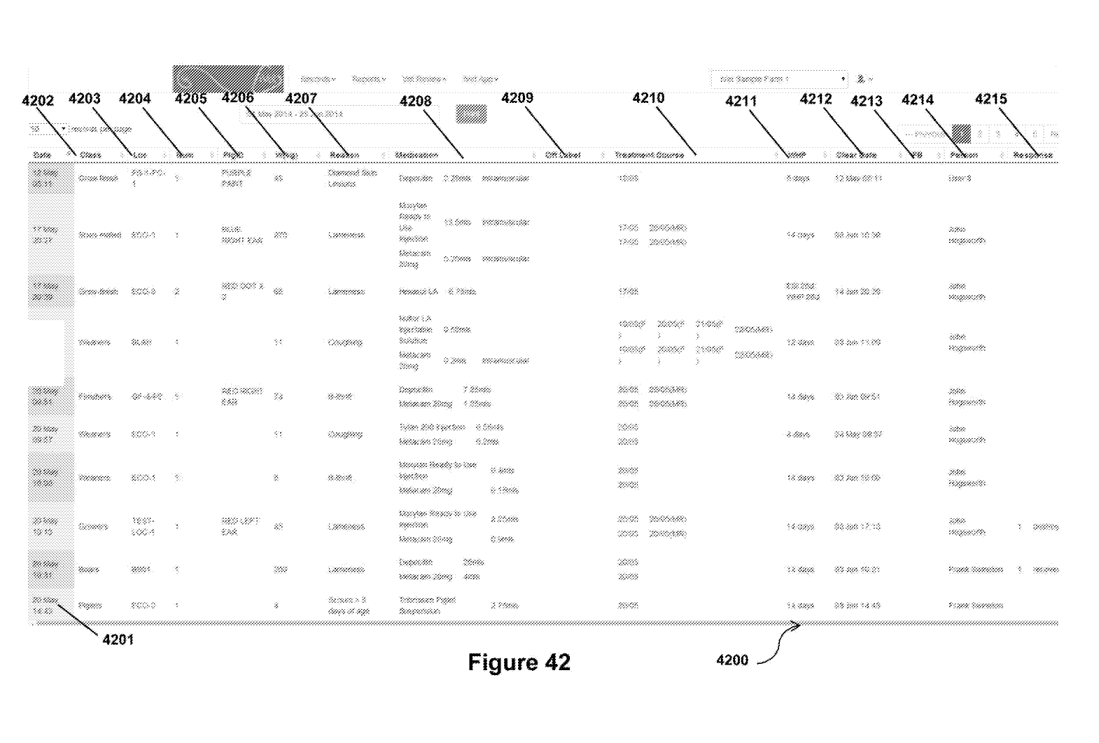

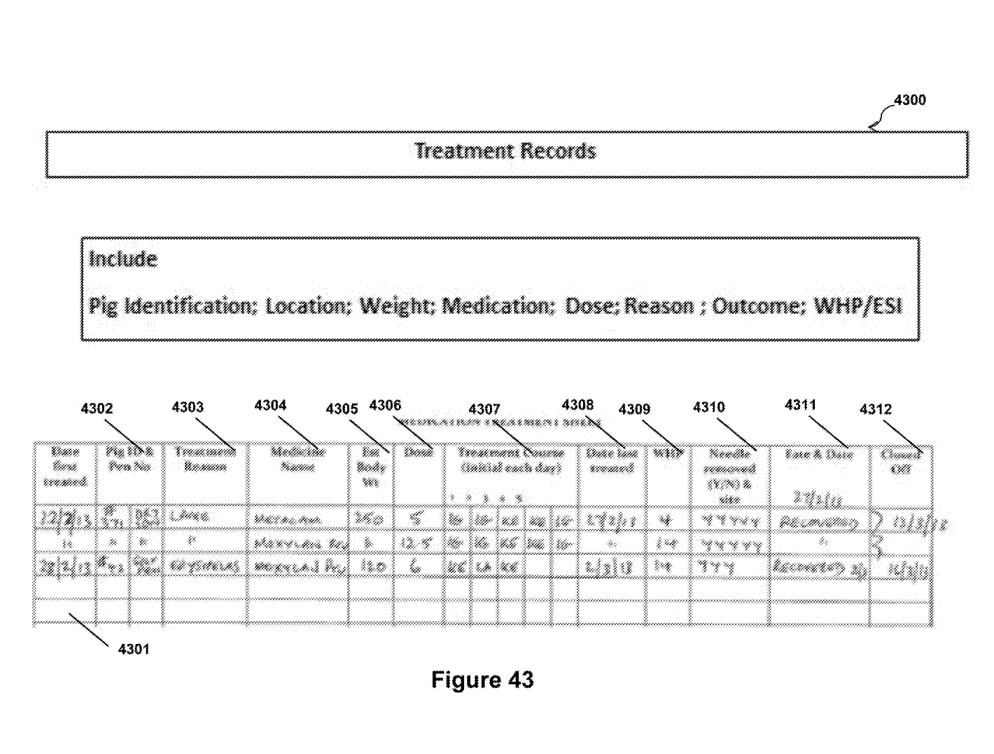

FIG. 42 illustrates a report showing records of treatment in a format that is compliant with regulatory bodies within an adjustable time period. It shows date 4201, class, 4202, location 4230, number treated 4204, id 4205, weight 4240, reason 4207, medication 4208, whether the use is off-label 4209, treatments 4210, WHP 4211, clearance date 4212, foreign body presence 4213, person 4214 and response 4215. For treatments that are not administered by the person who initiated the treatment, their initials are recorded against the treatment course 4210. If the dose administered varies from that prescribed, it is recorded in the treatment course 4210. FIG. 43 shows an example of a current paper based representation of similar regulatory compliant record of treatment.

In a preferred form, after using methods detailed above, a healthcare professional is able to adjust the veterinary health plan for a farm.

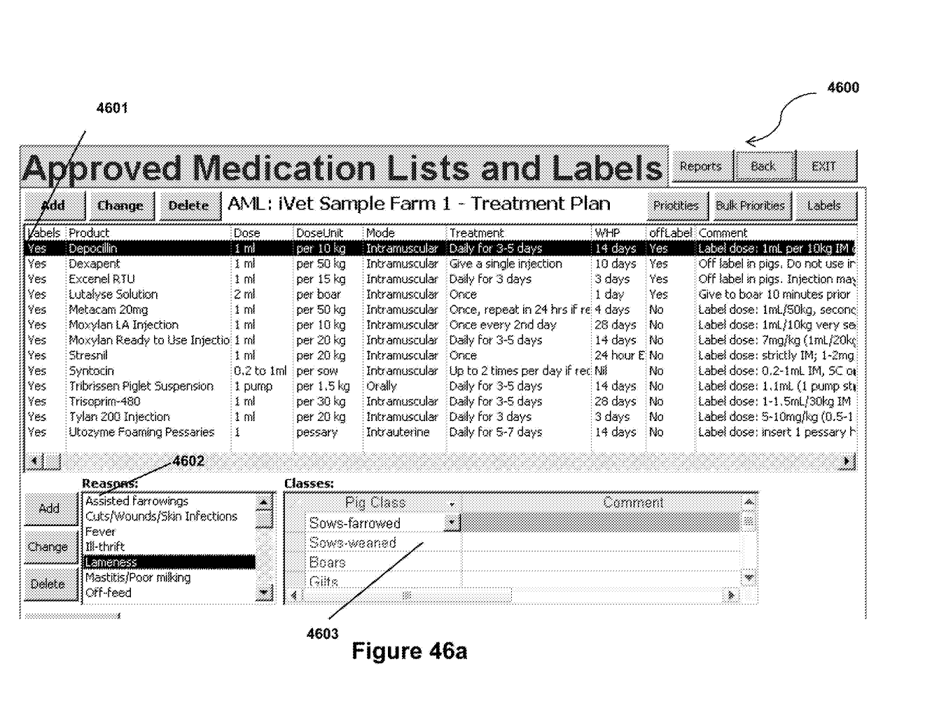

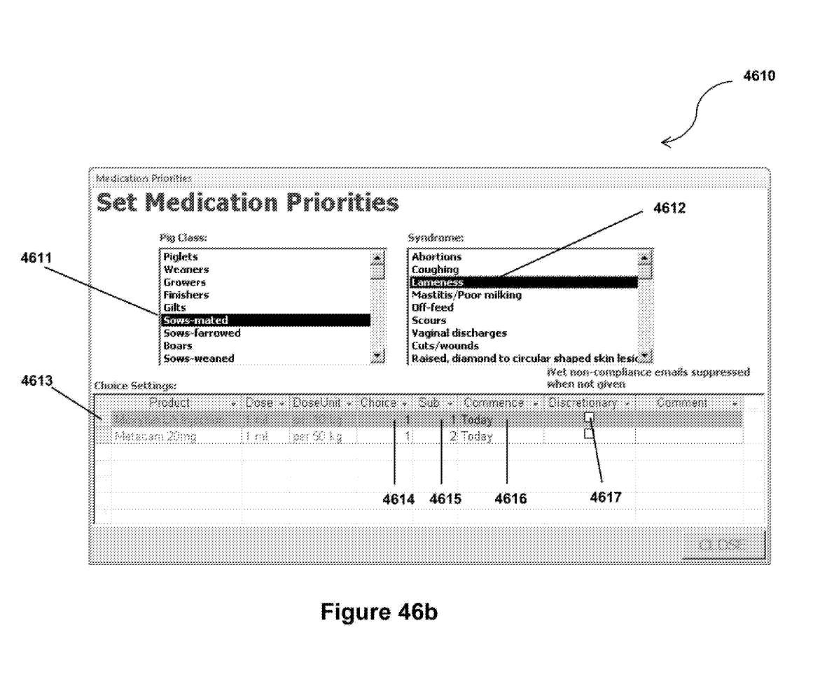

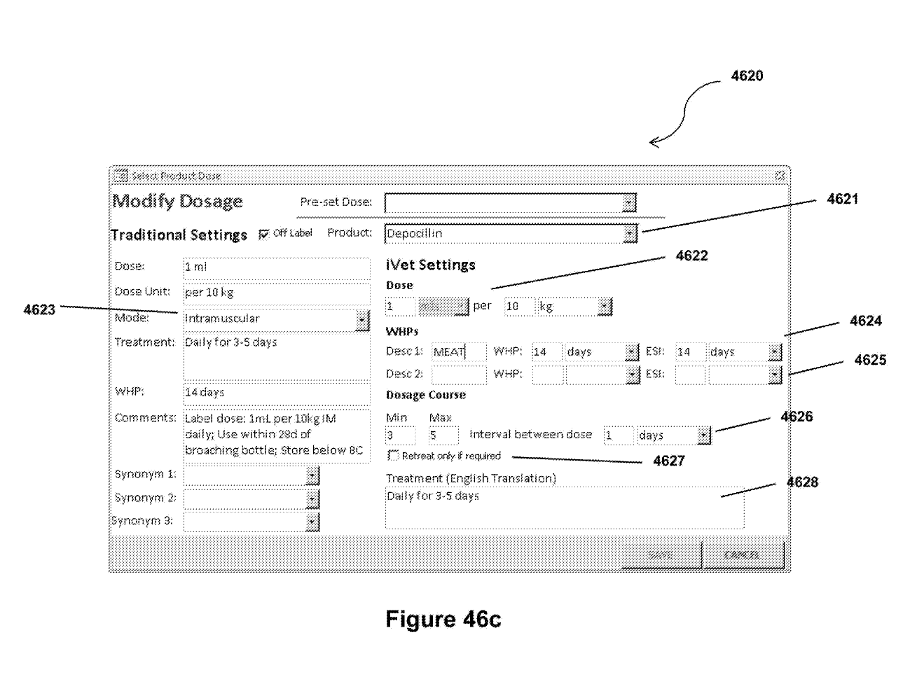

FIG. 46a shows a method for detailing the veterinary health plan for individual treatments via the Approved Medication Planner (AML) database. FIG. 46c shows detail for a specific medication. Where multiple medications are recommended for a particular class 4603 and reason 4602 they are configured as illustrated in FIG. 46b and their priorities set 4614 and 4615. A medication can also be flagged as discretionary 4617 which suppress notifications relating to omission of administration.

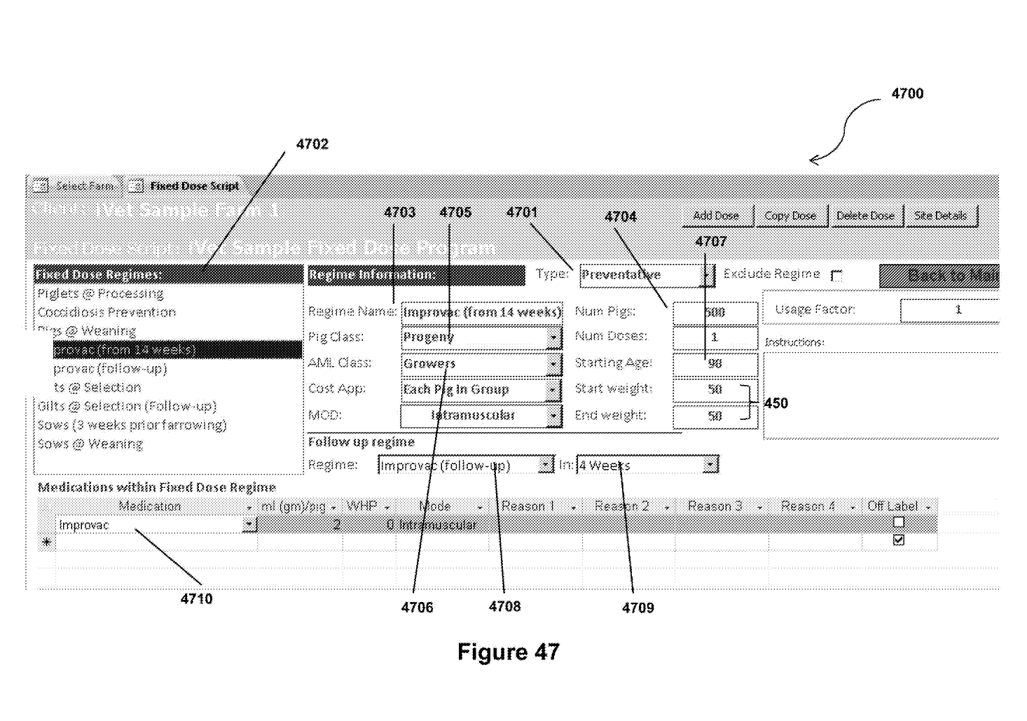

FIG. 47 shows a method for detailing the veterinary health plan for routine treatments. Of note, the healthcare professional is able to specify class 4706, medications and reasons 4710 as well as a follow-up routine regime 4708 and 4709 which creates a sequence of routine treatments.

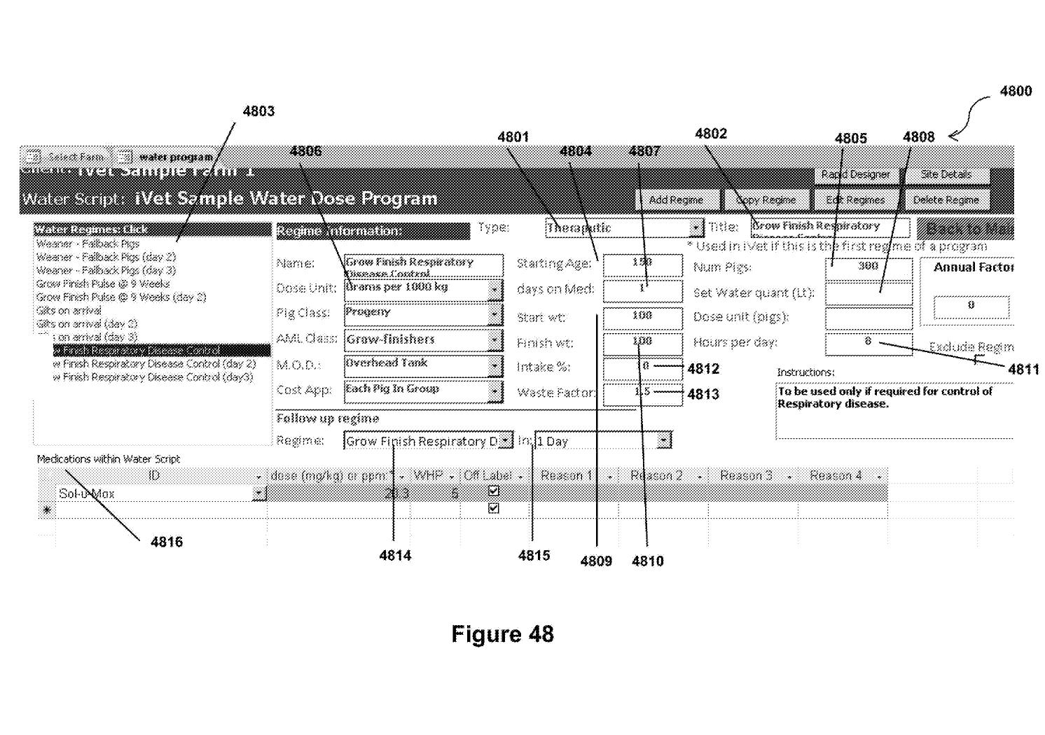

FIG. 48 shows a method for detailing the veterinary health plan for group treatments. Of note, the healthcare professional is able to specify type 4801 as well as a follow-up group regime 4814 and 4815 which creates a sequence of group treatments.

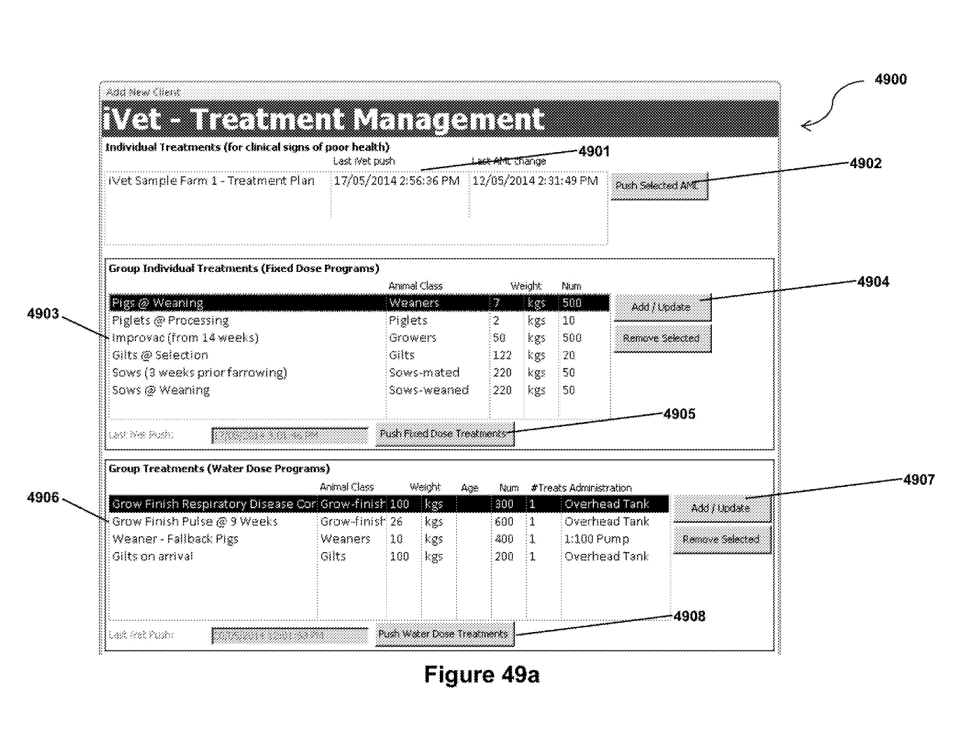

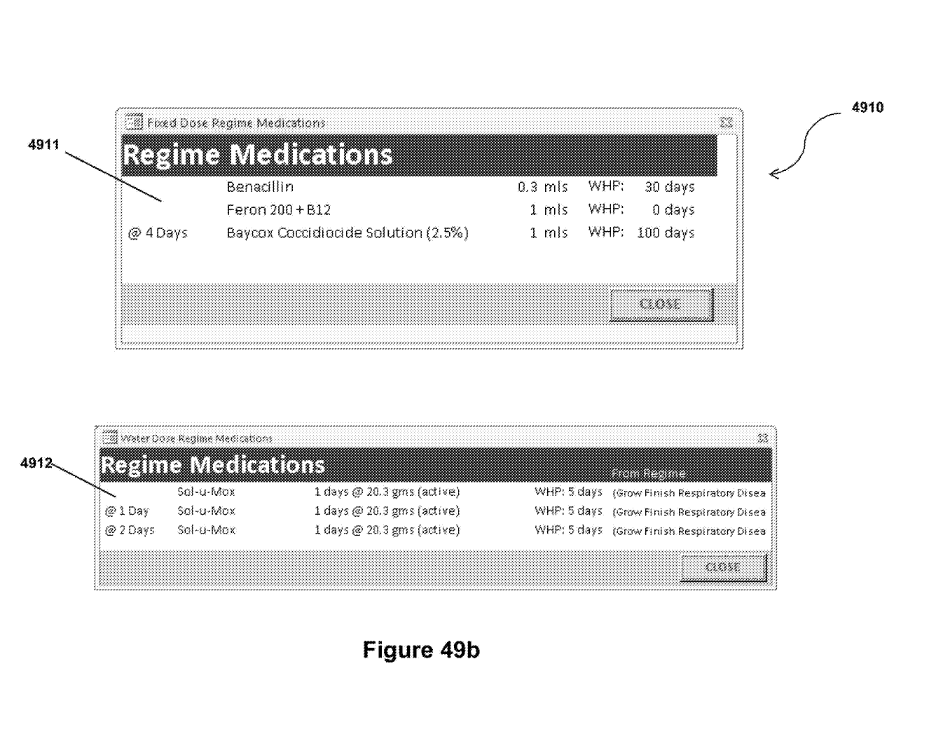

Treatment plans are able to be delivered to the users at terminals 105, 110 and 115 via the method illustrated in FIG. 49a.

Changes to an individual treatment plan can be delivered by clicking 4902, routine treatments by clicking 4905 and group treatments by clicking 4908. Medications within a plan can be viewed and are illustrated in FIG. 49b.

FIG. 2c is a database diagram 131a showing the structure of the Approved Medication Planner (AML) database recorded on for example the server 125 of FIG. 1. The database 130 is a master data store from which treatment plans for the user are determined based on the measurement data from investigations by the healthcare professional.

For example, the database 130 includes classes of animal 226 and reasons for treatment 224. A farm within the system 221 can have any number of individual treatment protocols 222 which include attributes such as medication, dose rate, instructions and withholding period. These protocols pertain to a number of individual treatment reasons 223, which relate back to a reason for treatment 224. An individual treatment reason 223 is applicable to a number of individual treatment reason classes 225 which are assigned treatment priority via attributes treatmentOptionNum and treatmentSubOptionNum. Individual treatment reason classes 225 relate back to a class of animal 226. A class of animal has a broader overviewing category 227.

In database 130, a farm 221 can have any number of routine treatment protocols 228 which include attributes such as medication, dose rate, withholding period, instructions and a scheduled date. A routine treatment is given for a number of routine treatment reasons 229, which relate back to a reason for treatment 224. A routine treatment 228 is also applicable to a single animal class 226. A routine treatment 228 can be immediately followed by another routine treatment 228 to form a sequence of routine treatments.

In database 130, a farm 221 can have any number of group treatment protocols 230 which include attributes such as medication, dose rate, withholding period, instructions and a scheduled date. A group treatment is given for a number of group treatment reasons 231, which relate back to a reason for treatment 224. A group treatment 230 is also applicable to a single animal class 226 and a group treatment type 232. A group treatment 230 can be immediately following by another group treatment 230 to form a sequence of group treatments.

FIG. 2d shows a basic object diagram 131b representing a particular instance of database 130. The database 130 contains a single farm 241, two reasons for treatment 244 and 255 and two classes of animal 246 and 248.

Farm 241 has two individual treatment protocols 242 (Moxylan Ready to Use injection) and 250 (Dexason) which are given for the same underlying reason 244 (Crusty). Treatment protocol 242 (Moxylan Ready to Use injection) for reason 244 (Crusty) is applicable for classes of animal 246 (piglets) and 248 (growers). Treatment protocol 250 (Dexason) for reason 244 (Crusty) is only applicable for class of animal 248 (growers).

Farm 241 has a routine treatment sequence. The first treatment 253 (consisting of Improvac) is given to growers 248 for aggressiveness 255. It is then followed by another treatment of improvac 256 for the same reason 255 and animal class 248. Farm 241 does not have any group treatment protocols.

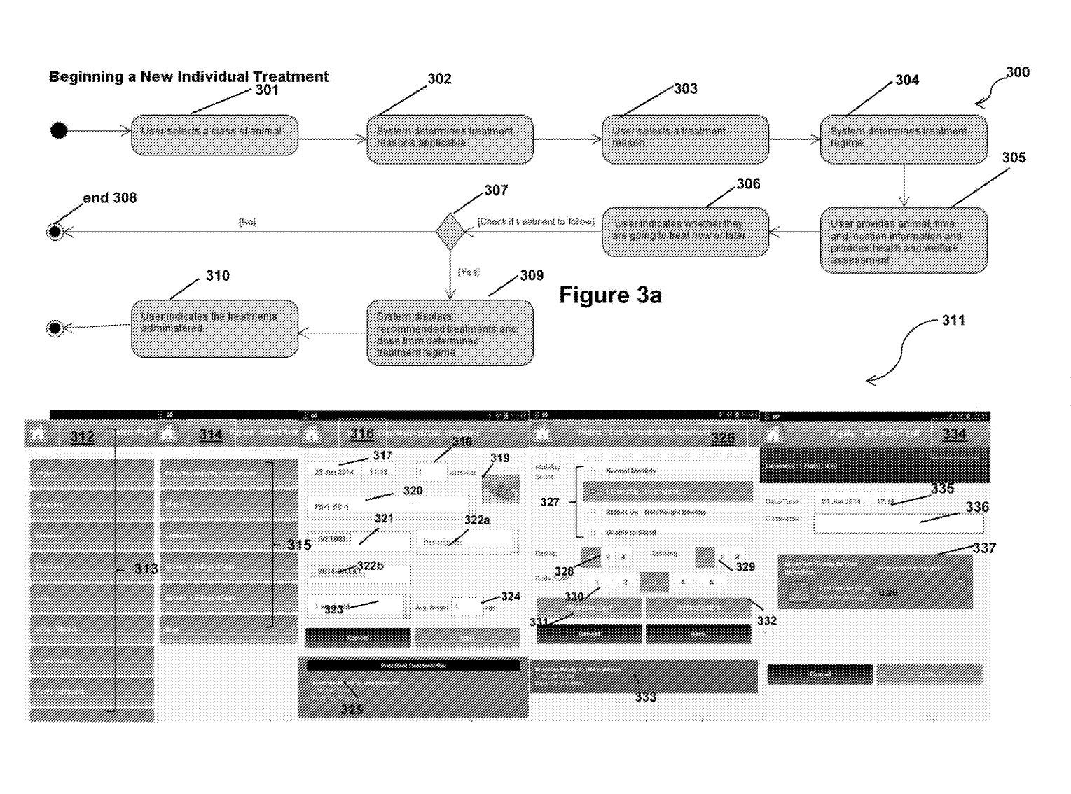

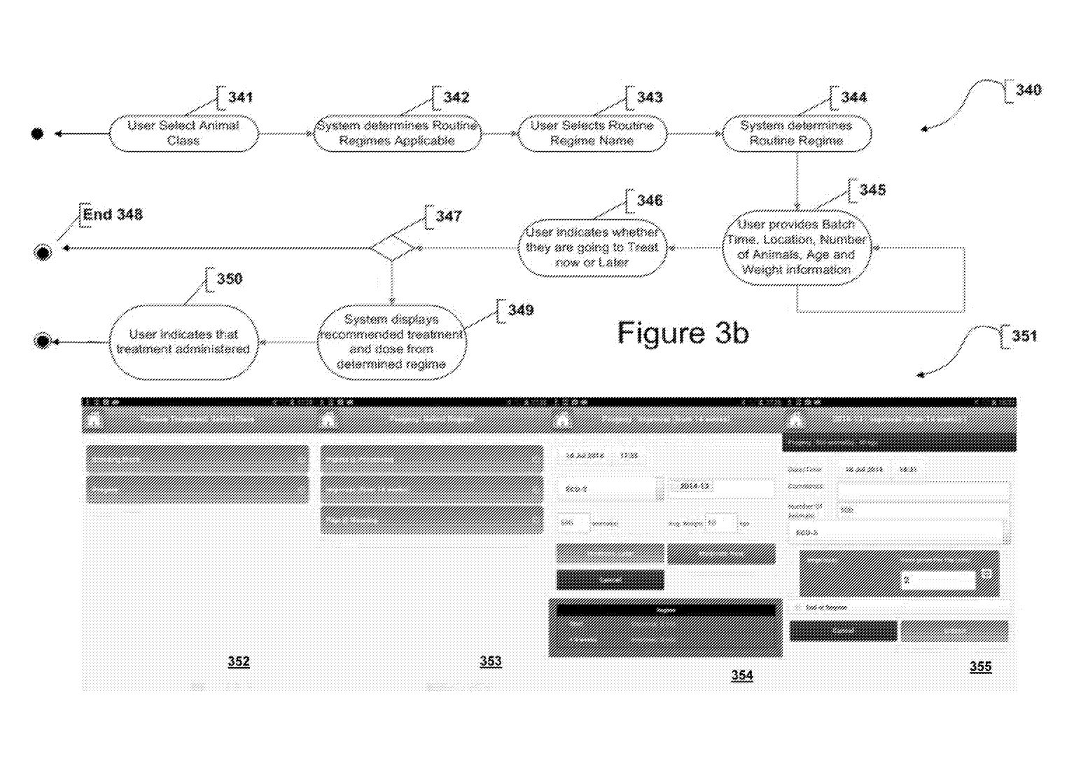

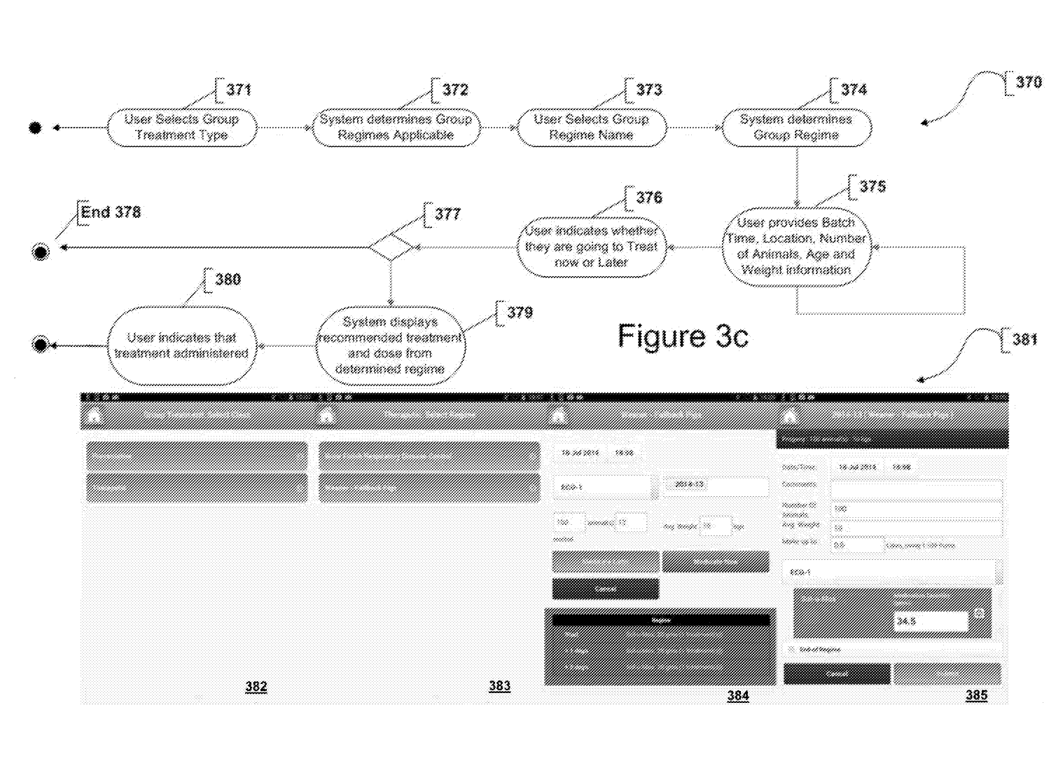

FIG. 3a is a flow diagram with corresponding example screen shots showing in further detail, the process for managing the health of one or more animals that require Individual Treatment. At step 301 the user associated with user device 105, 110, 115 (typically a farmer or trained stock hand) selects a class of animal 313, to which their problem relates. The user in this case may select from a number of pig classes including piglets, weaners, growers, finishers, etc. 312.

Selection of one of the options, 301 moves to step 302 in which the system determines treatment reasons which may be applicable to that particular class of animal determined at step 301. Treatment reasons are automatically generated from the veterinary health plan. At step 303 the user selects from the reasons 315 and control moves to step 304 where the system determines the treatment regime after which the system 100 via portable, user devices 105, 110, 115 provides input screens so as to capture measurement data. The input screens are presented to the user namely 316, 326 and 334 and this information is provided at step 305. For example 316 illustrates a number of fields in which the user may fill out in relation to the condition of the animals including: the date and time 317, a photograph of the animal with the symptoms 319, the location of the animal 320 (via an appendable list box), the number of animals 318, a unique identifier associated with the animal(s) 321 an/or animal descriptor 322a (via an appendable list box), the animal batch 322b (via an appendable list box), the age of the animal(s) 323, the weight of the animal(s) 324 (which is prefilled if an age is specified).

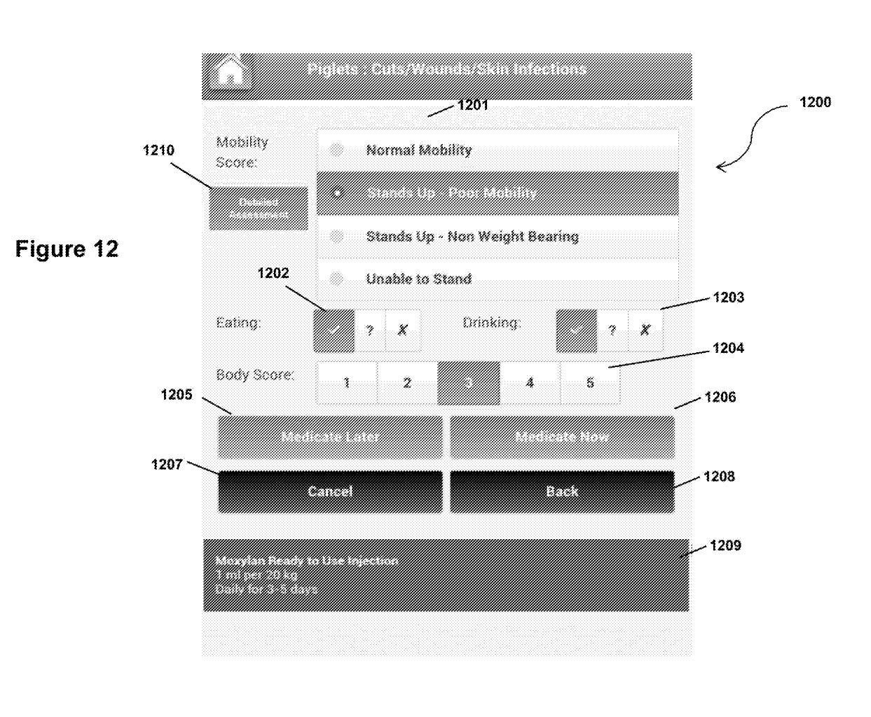

Further input screen 326 includes a health score 327 in which the user can select from one or more general health options related to the animal, whether or not the animal is eating 328, whether or not the animal is drinking 329, body score 330, the option to medicate later 331 or medicate now 332. The system 100 has provisions to insert add-on assessment modules (see 1210 in FIG. 12) enabling the user to supply additional assessment data to the healthcare professional which may be used to further refine prescribed treatment plans or to supply additional data for other ancillary purposes.

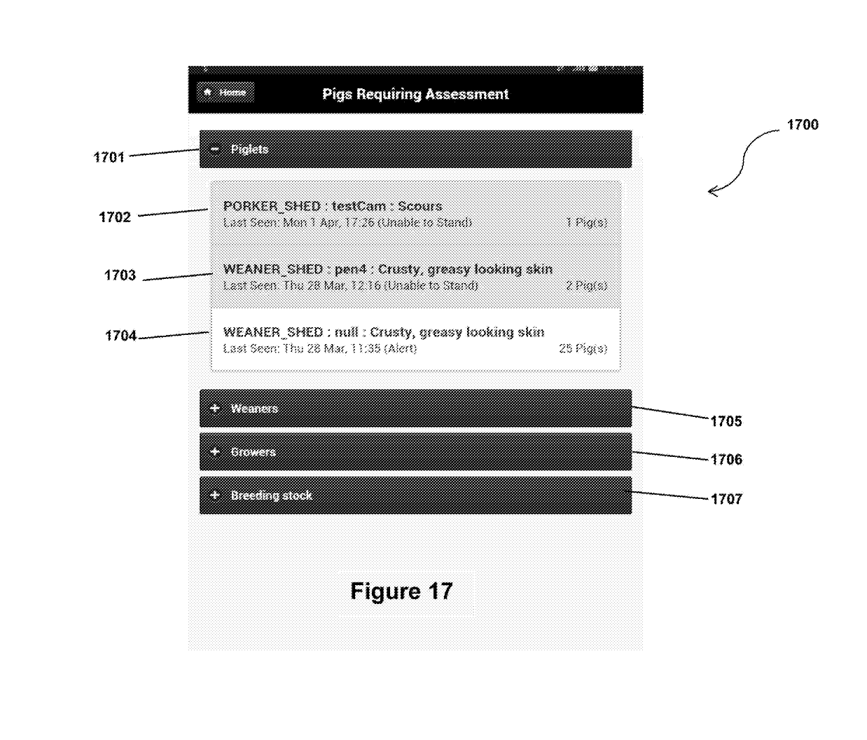

A user may not be equipped to administer the required treatment or the animal may just need to be monitored. The ‘medicate later’ option allows the system to identify the animal and place the record on an action list to help ensure the animal is treated albeit at a later time or so it can assessed again to review need for treatment. This action list is denoted “Animals Requiring Attention” (see FIG. 17). For example, this option enables the user to monitor individual animals with mild symptoms that may not warrant immediate treatment (e.g. umbilical hernia). At step 306, the user indicates whether they wish to undertake the treatment immediately 332 or at a later stage 331.

If the user elects to carry out treatment at a later stage the method ends at 308 and a reminder may be set up on the server 125 to remind the user to take action with regard to the treatment. Otherwise if at step 306 the user elects to carry out treatment immediately, control moves to 309 where the system displays recommended prescribed treatments from a determined treatment regime. The determined treatment regime is derived from user inputs from 312 and 314 with the potential for modification based on data acquired from 316 and 326.

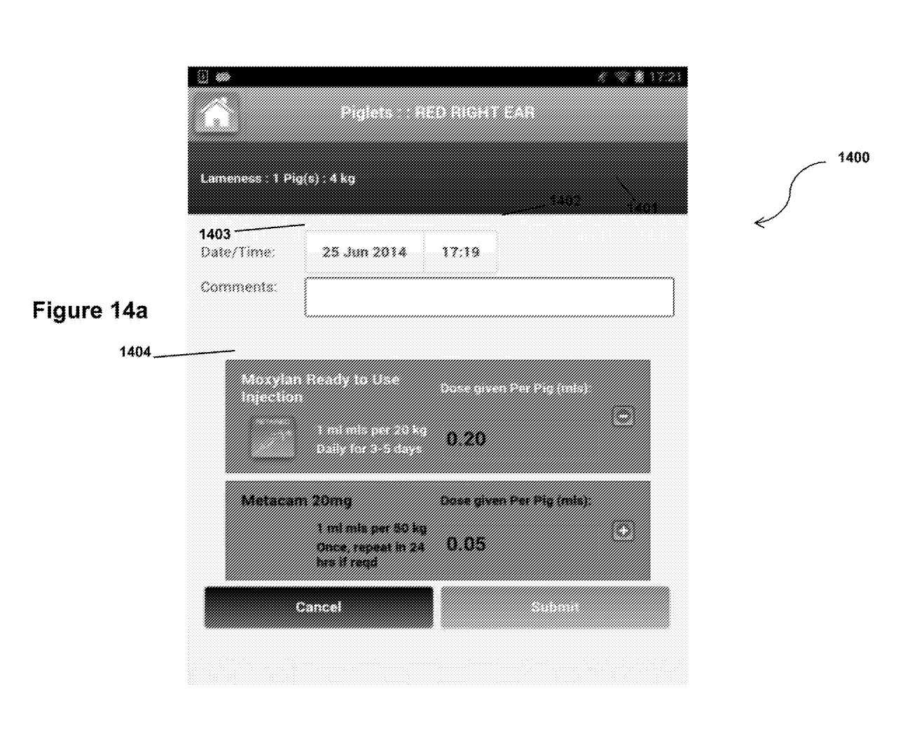

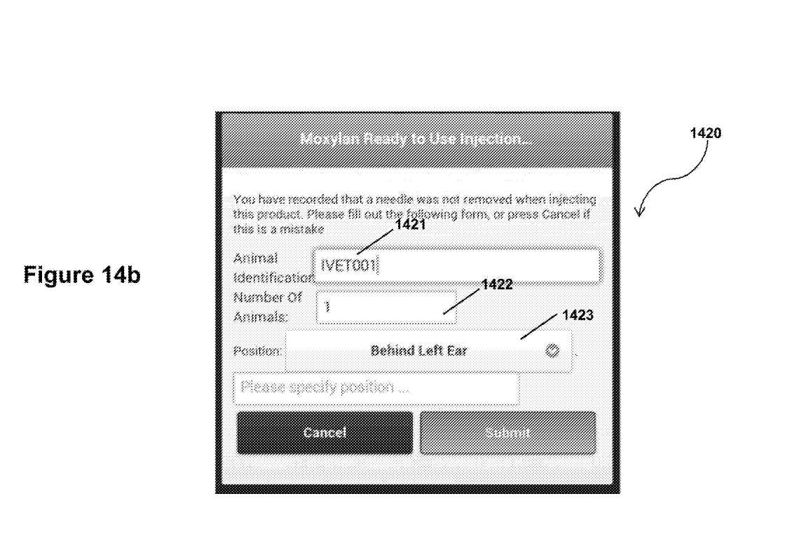

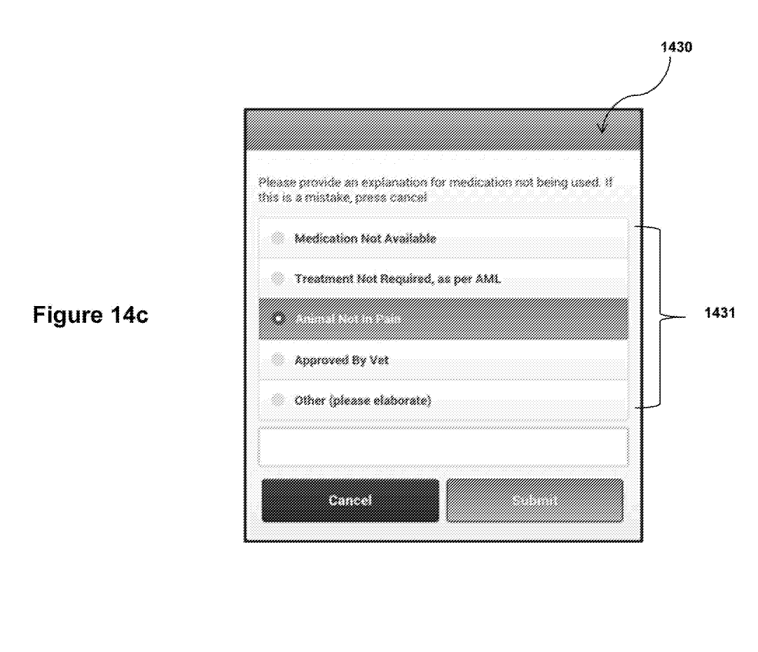

Screen shot 334 includes the date and time of treatment 335 (this defaults to the current time, however this can be adjusted where retrospective data entry is conducted) and an option to provide comments 336. Field 337 is a list of treatments recommended by the healthcare professional for this particular health event. The dose is automatically calculated using the dose rate within the treatment plan and the body weight specified at 324. The user indicates which treatments are to be actually administered by tapping the [+/−] button within 337. The system 100 has the capability to utilise barcodes and/or QR codes on medication container/packet so medication can be verified, tracked and accurate inventories determined, ensure expiry date compliance and for investigation of adverse reactions to medications (see 1408 in FIG. 14a). If required, users are able to adjust the dose actually administered. The consulting vet receives a notification where non-recommended doses are administered (see example screen shot FIG. 44a). If a medication of the prescribed treatment plan is not administered, the system 100 displays a list of reasons from which the user selects the appropriate option (see 1431 in FIG. 14c). Following the completion of this form, a notification is sent to the healthcare professional (see FIG. 44a).

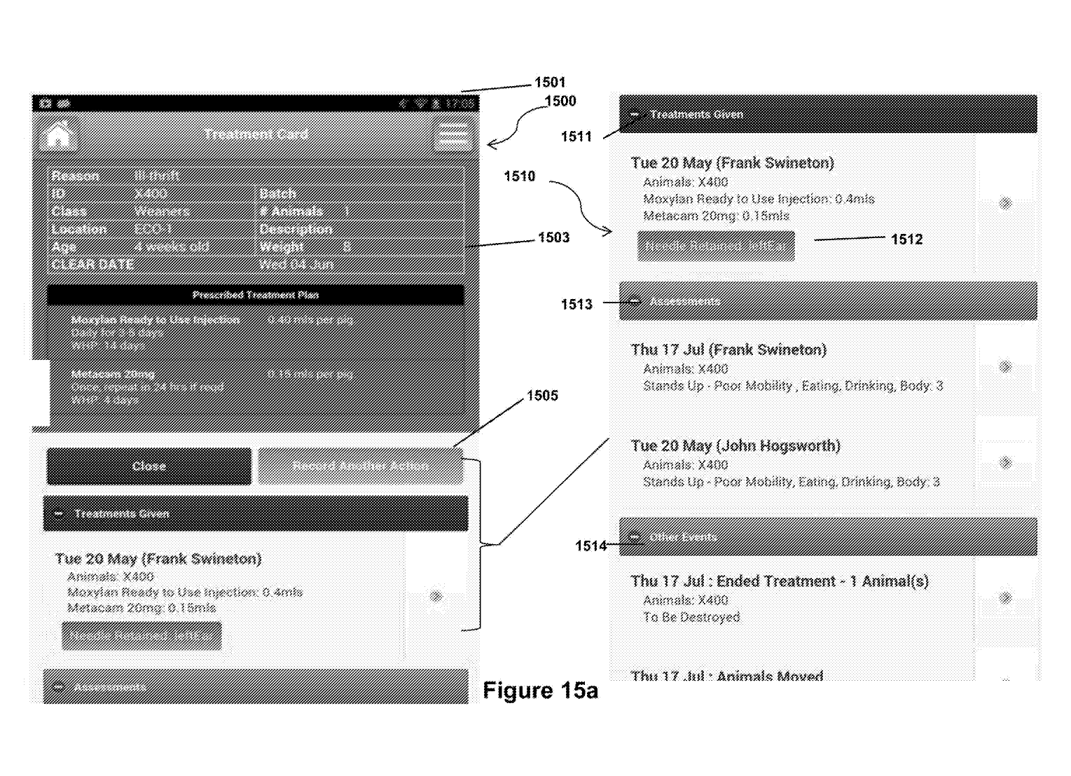

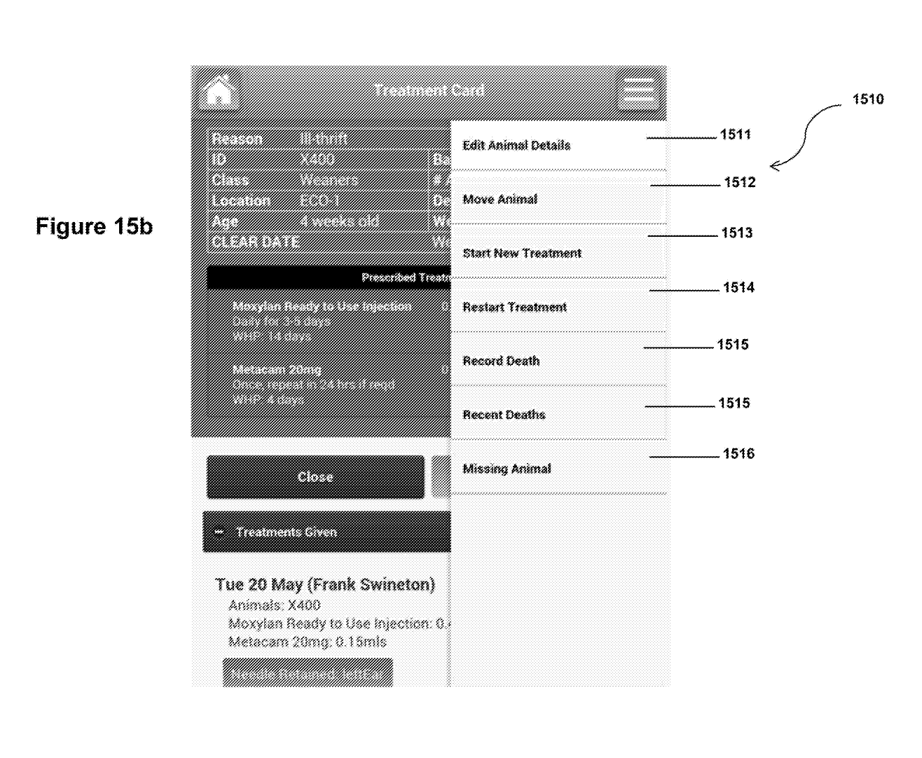

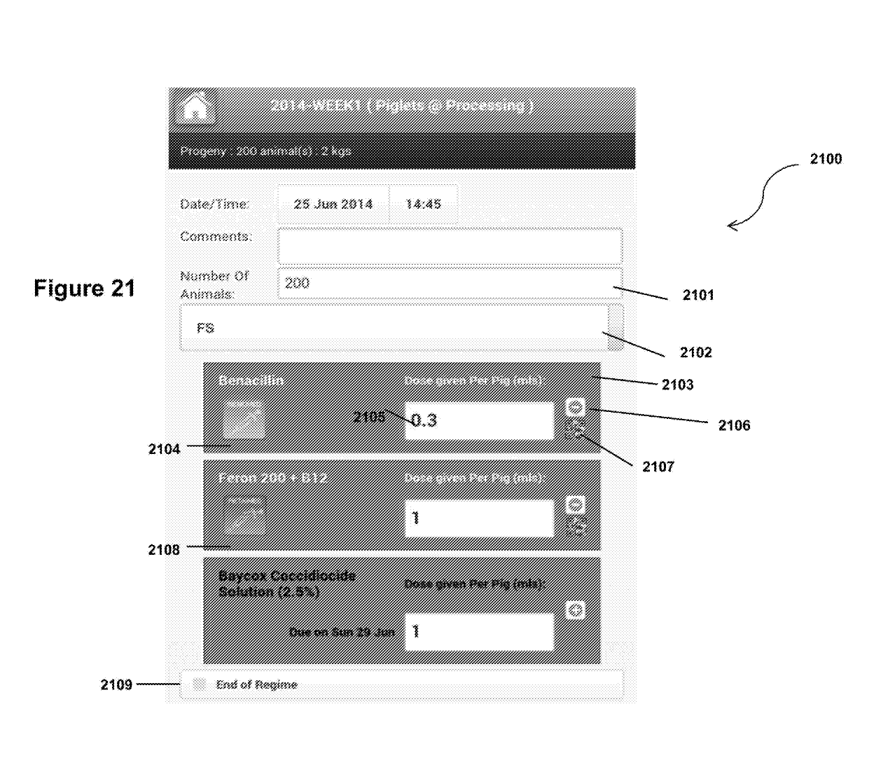

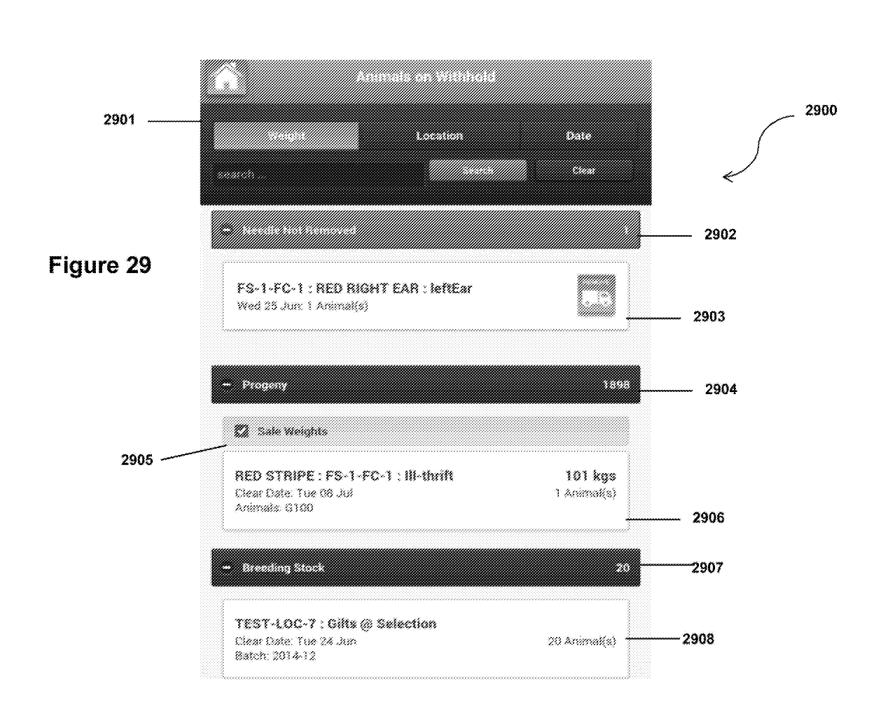

The system 100 has provision for the user to record the occurrence of a foreign body event (e.g. retained or lost needle) arising from administration of an injectable medication. The user selects the “needle” icon located at 337 which displays a data entry screen (1420 in FIG. 14b) to enter the following details: animal identification 1421, number of animals involved 1422, position of foreign body on animal 1423, or lost in environment. The user can toggle foreign body status, indicating that a hypodermic needle has been retained (see 2108 in FIG. 21) or removed (see 2104 in FIG. 21) in an animal. A positive foreign body status is recorded and viewable on the Treatment Card (see 1507 in FIG. 15a), and on the Withhold List (see 2902 in FIG. 29). Animals with a positive foreign body status remain on the Withhold List until they are removed from the herd.

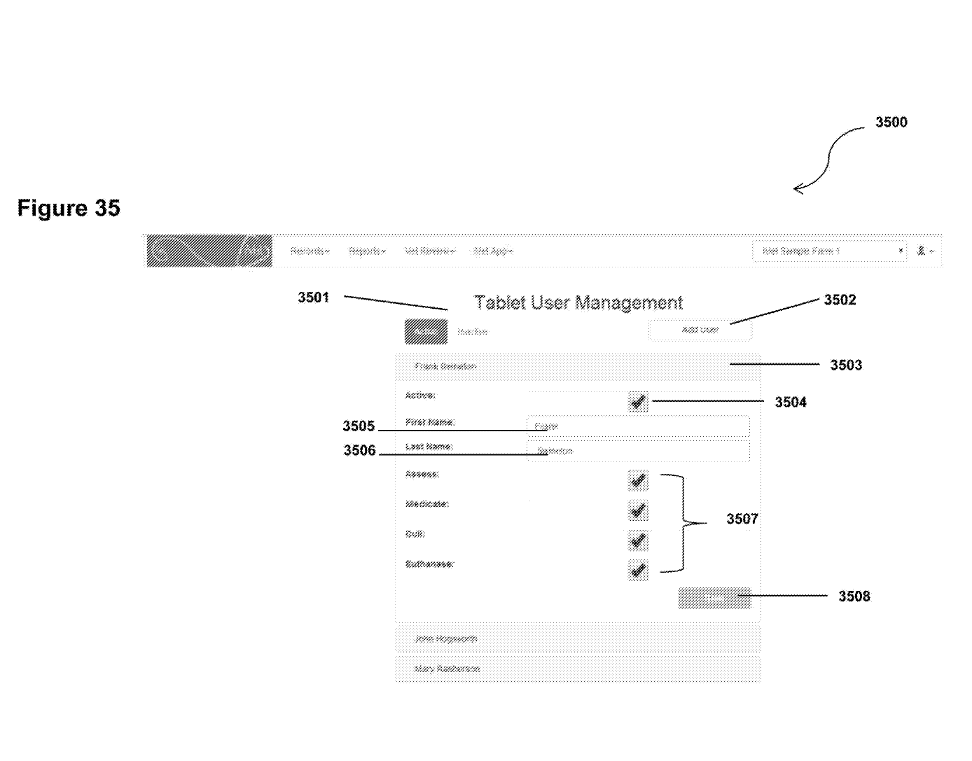

Control then moves to step 310 of FIG. 3a in which the user indicates that the treatments have been administered. The user administering the treatment is selected from a list of users deemed competent. The user of the system will be a competent stock person as determined by the consulting healthcare professional and the manager. The user will require to be trained and subsequently demonstrate competency in the use of the system to record the appropriate details, to identify and assess animals with health conditions, to medicate animals, to determine disposition of animals (such as to be culled or to be euthanized) and in the process of performing culling and euthanizing of animals. Acceptance that the user is deemed competent in these areas is recorded in the User Management screen (see FIG. 35).

The system 100 generates action lists to assist the user to manage animals currently under treatment (denoted “Animals Currently Treated”) and animals (those on treatment and those identified with a health condition) that require their daily health assessment (denoted “Animals Requiring Attention”). System 100, where pre-determined criteria are met may create an alert to follow up with the user and healthcare professional following the treatment at a predetermined time after the treatment to help ensure subsequent treatments are administered as specified in the treatment plan and to determine whether or not the treatment was successful.

The Animals Requiring Attention List (see FIG. 17) on the user device allows a user to obtain the numbers of animals requiring attention. The animals on the list are grouped by animal class, e.g. piglets 1702, weaners 1703. The user can see the location, the symptom and the last time that the pig was seen and whether or not the record is highlighted to alert the user to the animal indicating that it may require additional care (based on health scores) 1706. The records may be sorted by animal identification, location and date of last assessment 1701. The user is also able to search the records on these items. This sorting and filtering functionality exits for all screens displaying lists of records.

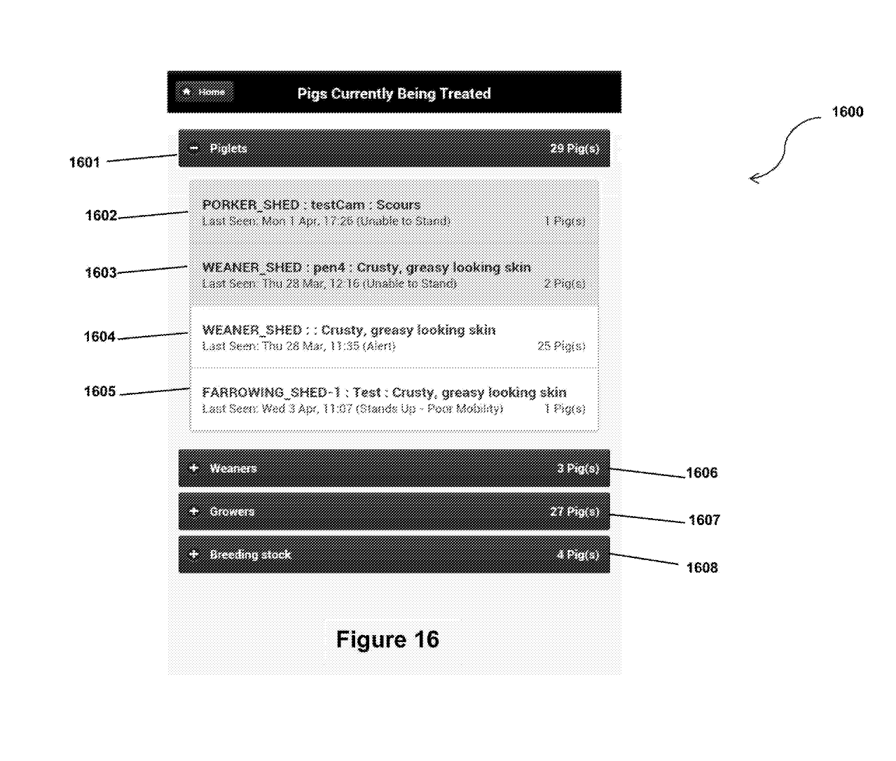

The Animals Currently Treated list (see FIG. 16) on the user device allows a user to review all animals currently being treated. The details displayed and sorting functionality is similar to those on the “Animals Requiring Attention” list.

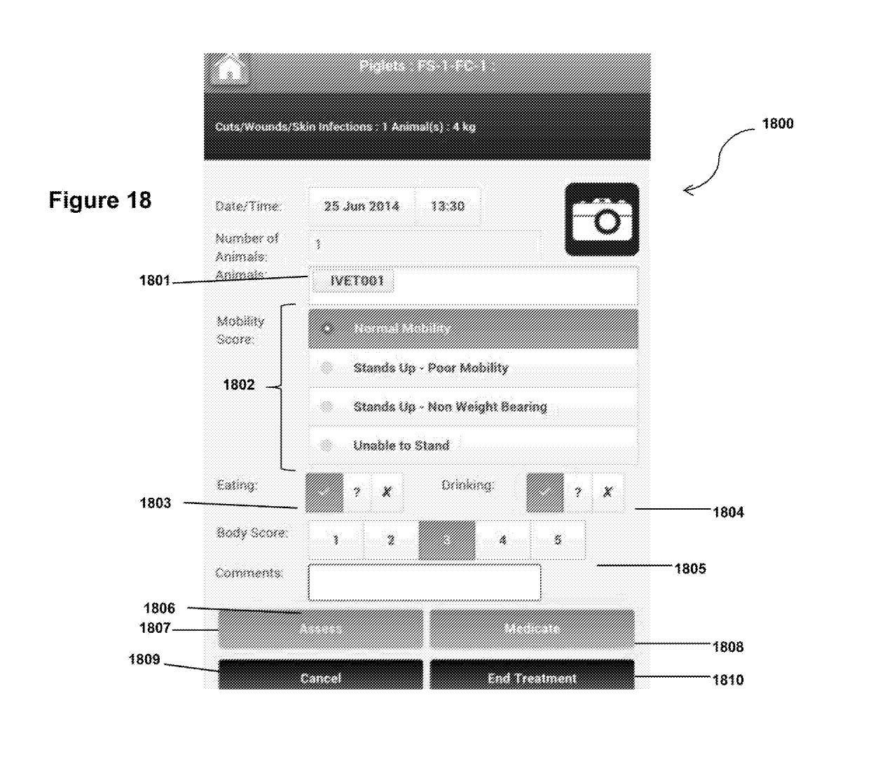

From the animal record located on the “Animal Currently Treated” or “Animals Requiring Attention” lists, the user proceeds to the Treatment Card of the animal (see FIG. 15a). The treatment card categorises the historical information regarding the animal by treatments given 1511, assessments 1513 and other events 1514 (animal movements, culls, euthanasia and deaths) for this particular health event for the animal. Subsequent health assessments and treatments are entered by the user by selecting “Record Another Action” 1505. This action brings up Animal Assessment form (see FIG. 18). The user enters health assessment data then the user may enter the treatment data. This process continues for the animal until the treatment regime is completed or the animal dies.

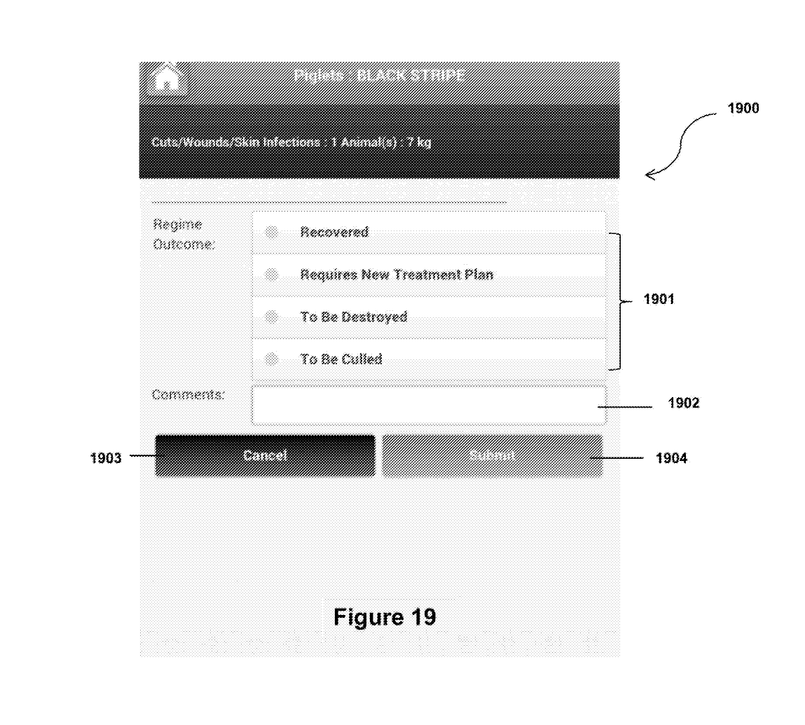

To complete a treatment regime (or to stop the monitoring an animal with a health condition not requiring medication), the user is required to enter an “End Treatment” event which is accessed from the Treatment Card 1810 after animal assessment data 1802, 1803, 1804 and 1805 has been entered. FIG. 19 displays the end treatment screen display 1900.

End Treatment events include the following types: recovered, requires new treatment plan, to be destroyed, to be culled, died or missing. The user selects the appropriate end treatment type 1900 to end the treatment which subsequently removes these animals from action lists 1600 and 1700.

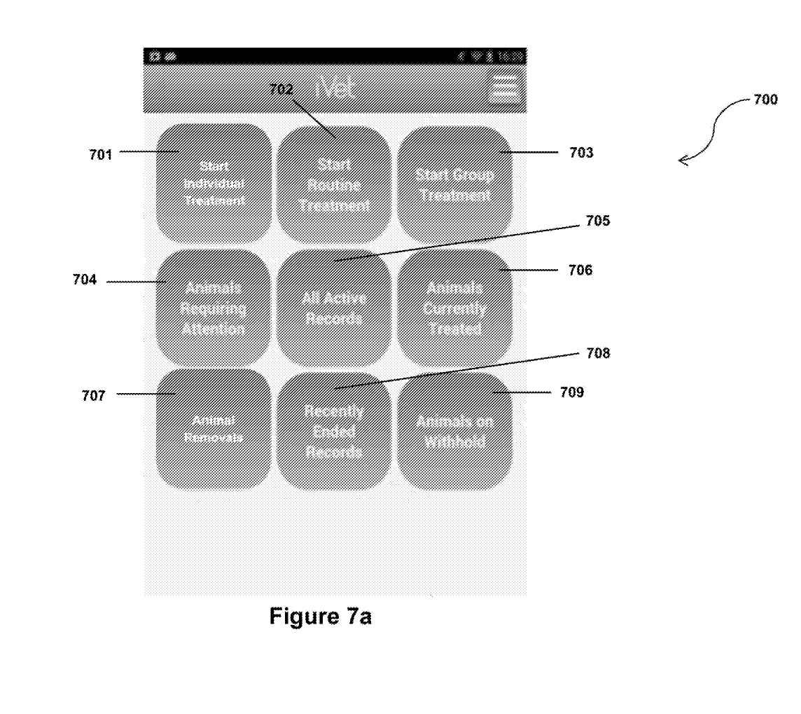

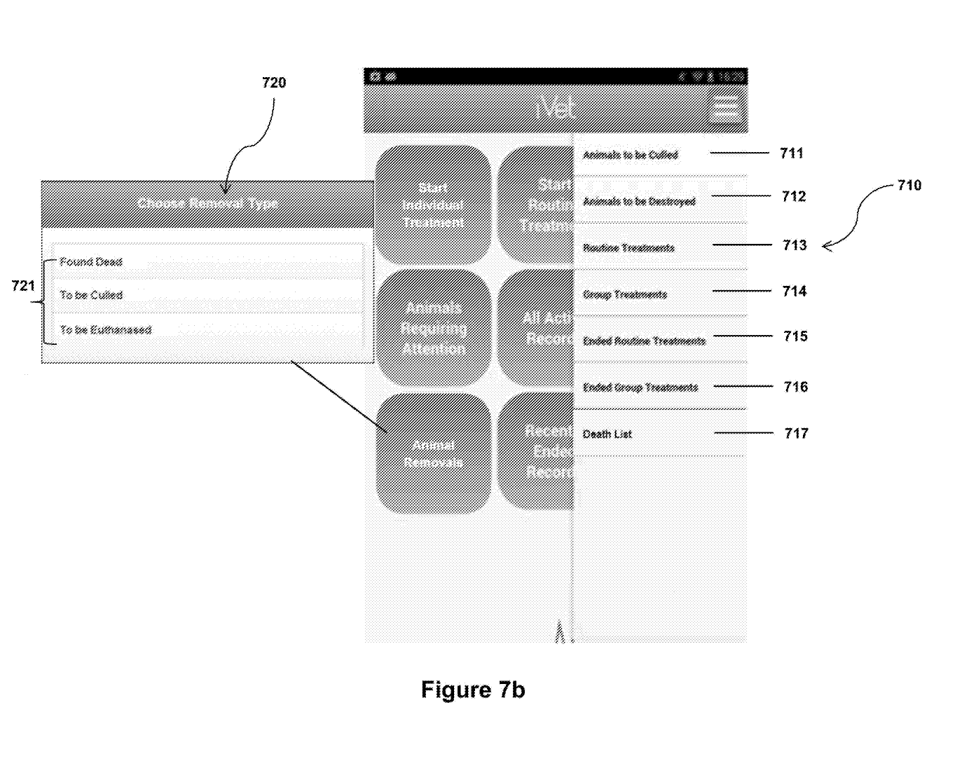

Animals that are given an End Treatment Status of “To be culled” or “To be Destroyed (euthanized)” require to be formally removed from the population from the menu on the Home screen (see 711 and 712 in FIG. 7b). These animals are selected from the “animals to be culled” 3005 and “animals to be euthanized” 3105 lists. The “Cull Selected” button 3003 or “Euthanize Selected” button 3102 is then selected which brings up the shortened list of the animals to be removed 3010, 3110. The animals are then submitted for removal from the population 3015, 3115.

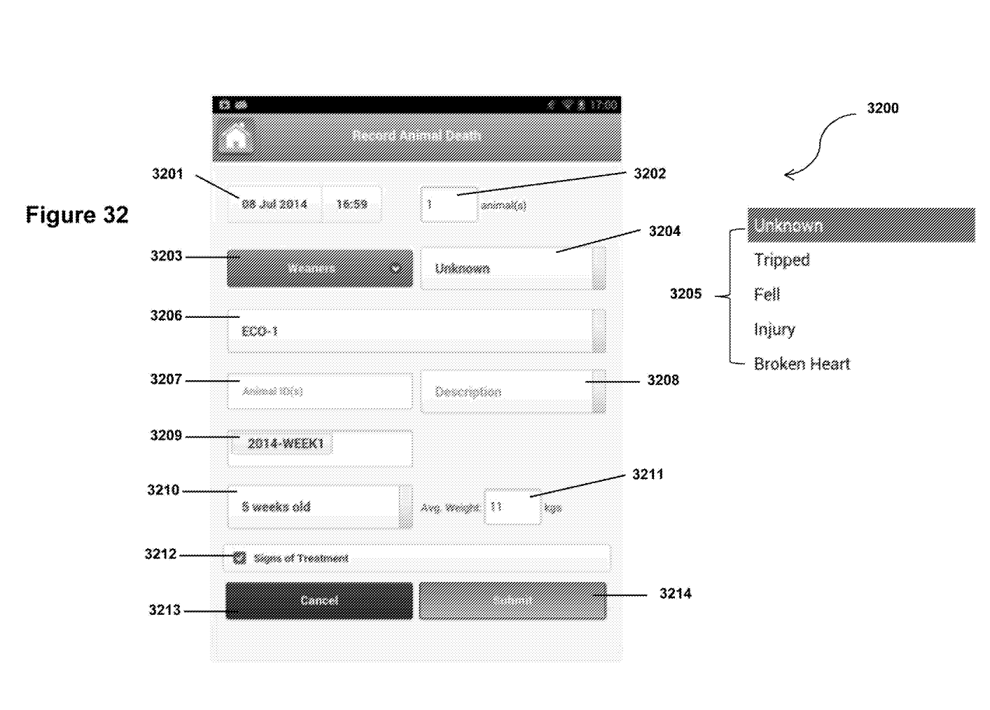

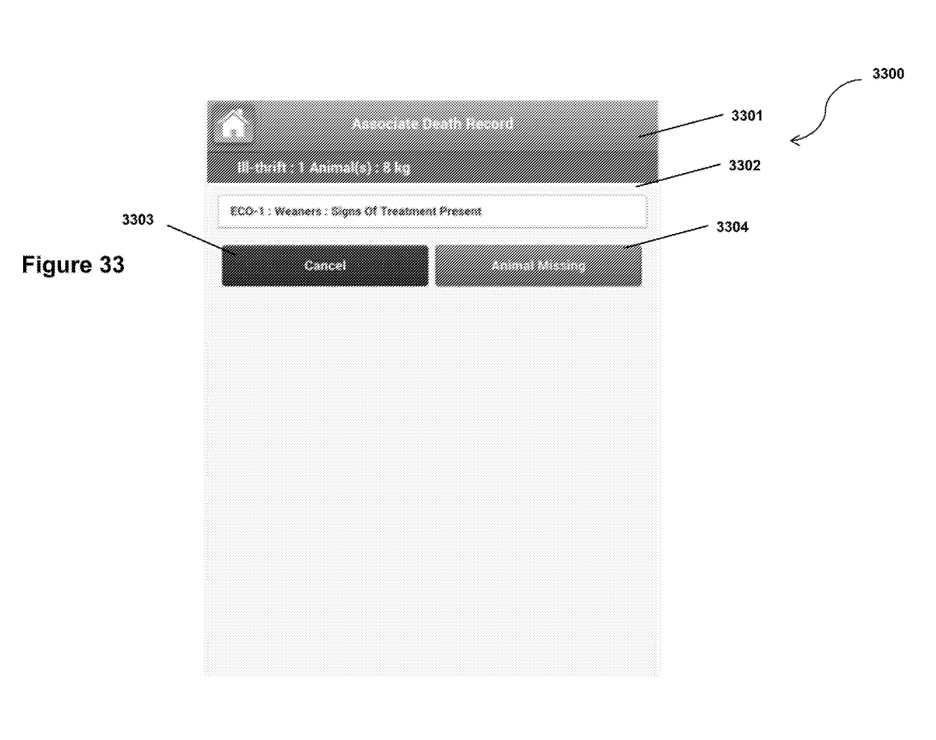

As animal assessments are not conducted on missing or dead animals, these end treatment events are not entered using 1900. Instead, these end treatment event types are entered from the menu of the Treatment Card (see 1515 and 1516 in FIG. 15b). After selecting “Record Dead”, the user enters data pertaining to the dead animal(s) FIG. 32. After selecting “Missing Animal”, the user is presented with a recently removed animal list (see FIG. 33) which may be associated with the respected pig death.

The system 100 allows users to enter the following animal events that do not relate to animals currently on treatment or being monitored for a health condition: deaths, euthanasia and culling. These events are entered from the “Animal Removals” button (see 707 in FIG. 7a). The user then selects the appropriate event from the options (see 721 in FIG. 7b). FIG. 32 shows an example of the data entry form and data entered by the user when entering a death: current date and time 3201, animal(s) identification (unique) 3207, animal(s) descriptor (a non-unique identifier) 3208, animal batch 3209, number of animals 3202, age 3210, weight 3211, location 3208, animal class 3230 and reason for death 3204 & 3205 and sign of treatment 3212. After the user submits the data 3214, the animals go onto the death list database 717, and the recent death list 1515. The animals listed on the recent death list can be associated to a treatment record via the missing animal functionality (see 1516 in FIG. 15b). Similar functionality is available for animals that are to be culled and are to be euthanized.

A Routine Treatment Plan involves the administration of a set amount of medication to each animal within a population irrespective of body weight. FIG. 3b is a flow diagram with corresponding example screen shots showing in further detail, the process for managing the health of one or more animals that require Routine Treatment. At step 341 the user associated with user device 105, 110, 115 (e.g. typically a farmer or trained stock hand) selects a class of animal 352, to which their problem relates. The user in this case may select from a number of pig class categories including breeding stock and progeny 352.

Selection of one of the options 341 moves to Step 342 in which the system determines Routine Treatment Plans which may be applicable to that particular class of animal determined at step 341. Routine Treatment Plans are automatically generated from the veterinary healthcare plan once the animal class is provided. Routine Treatment Regimes can be linked together and therefore create a sequence of Routine Regimes to be scheduled for a batch of animals via the Approved Medication Planner (AML) (see 4708 and 4709 in FIG. 47).

At step 343, the user selects the appropriate Routine Treatment Plan. The user in this example may select from the list of the following Routine Treatment Plans: Piglets @ Processing, Improvac (from 14 weeks) and Pigs @ Weaning (see screen shot 353). Control then moves to step 344 where the system 100 via user devices 105, 110, 115 determines the Routine Treatment Plan. Control then moves to 345 which provide input screens 354 so as to capture measurement data about the group of animals to be treated, namely date and time 2001, location 2002, Batch ID 2003, number of animals 2004, average age and average weight 2005 of the group.

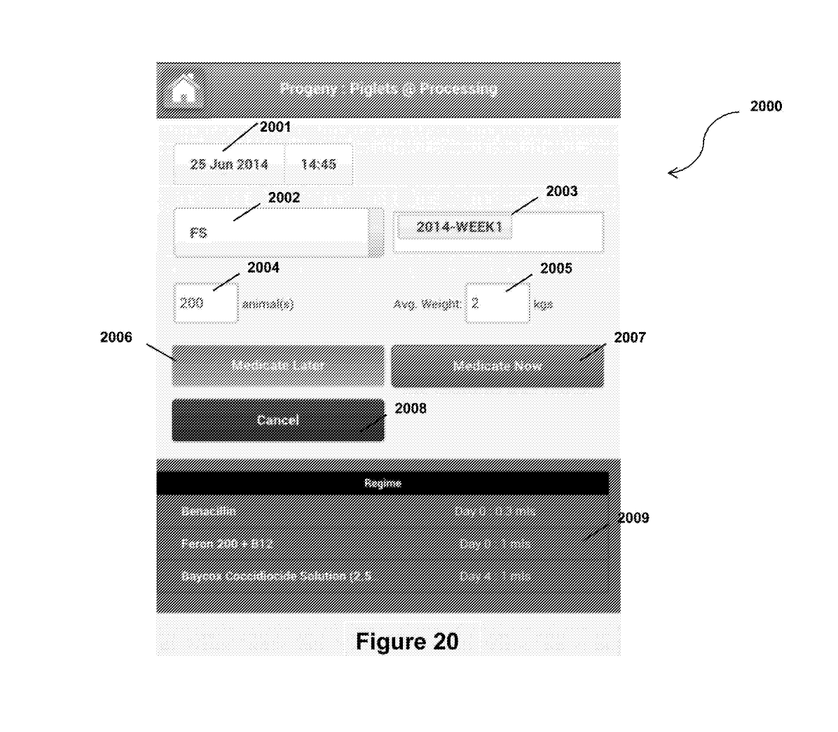

At step 346, the user indicates whether they wish to undertake the treatment immediately or at a later stage (see 354 and FIG. 20). A user may not be equipped to administer the required Routine Treatment. The ‘medicate later’ option allows the system to identify the batch of animals and place the record on an action list to be treated later (see FIG. 17). If the user elects to carry out treatment at a later stage the method ends at 348.

Otherwise if at step 346 the user elects to carry out treatment immediately, control moves to 349 where the system displays recommended prescribed treatments from a determined Routine Treatment Regime 355 (and see 2103 in FIG. 21).

Screen shot 355 includes the date and time of treatment (this defaults to the current time, however this can be adjusted where retrospective data entry is conducted) and an option to provide comments. This screen specifies the Routine Treatments as recommended by the healthcare professional for this particular Treatment Regime in addition to listing the other Treatment Regimes linked to this Treatment Regime (see FIG. 21). The user indicates which treatments are to be administered by using the [+/−] button 2106. If required, the user can adjust the dose prescribed. The healthcare professional receives a notification where non-recommended doses are administered (see FIG. 44a). If a medication of the prescribed treatment plan is not administered, the invention displays a list of reasons from which the user selects the appropriate option (see FIG. 14c). Following the completion of this form, a notification is sent to the healthcare professional (see FIG. 44a).

The system 100 has provision for users to end a Routine Treatment Regime prematurely (see 2109 in FIG. 21).

Control then moves to step 350 of FIG. 3b in which the user indicates that the treatments have been administered. The user administering the treatment is selected from a list of users deemed competent. The system 100 may create an alert to follow up with the user following the treatment at a predetermined time after the treatment to help ensure subsequent treatments are administered as specified in the treatment plan and to determine whether or not the treatment was successful.

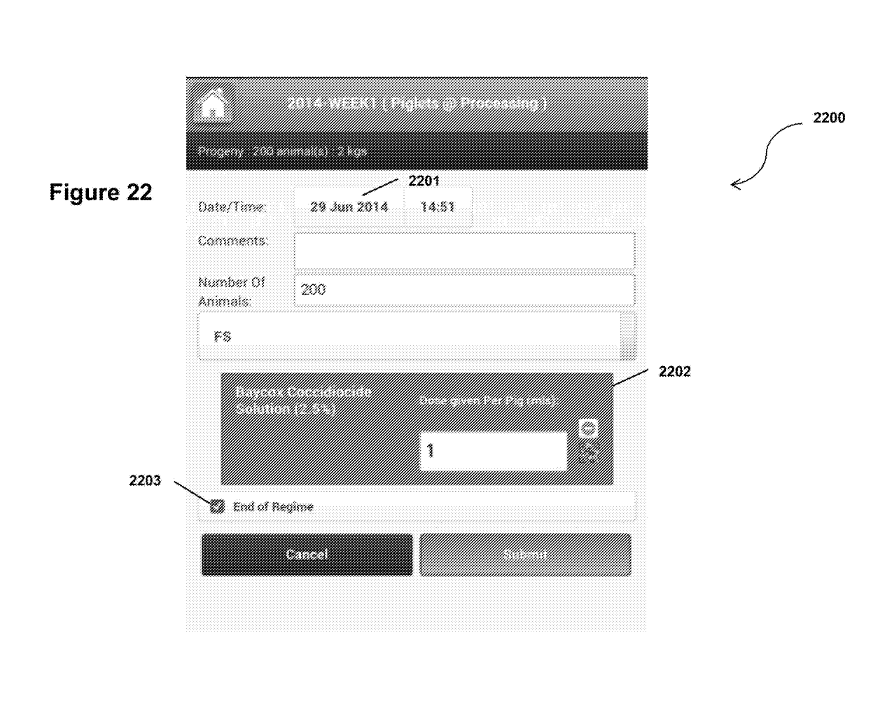

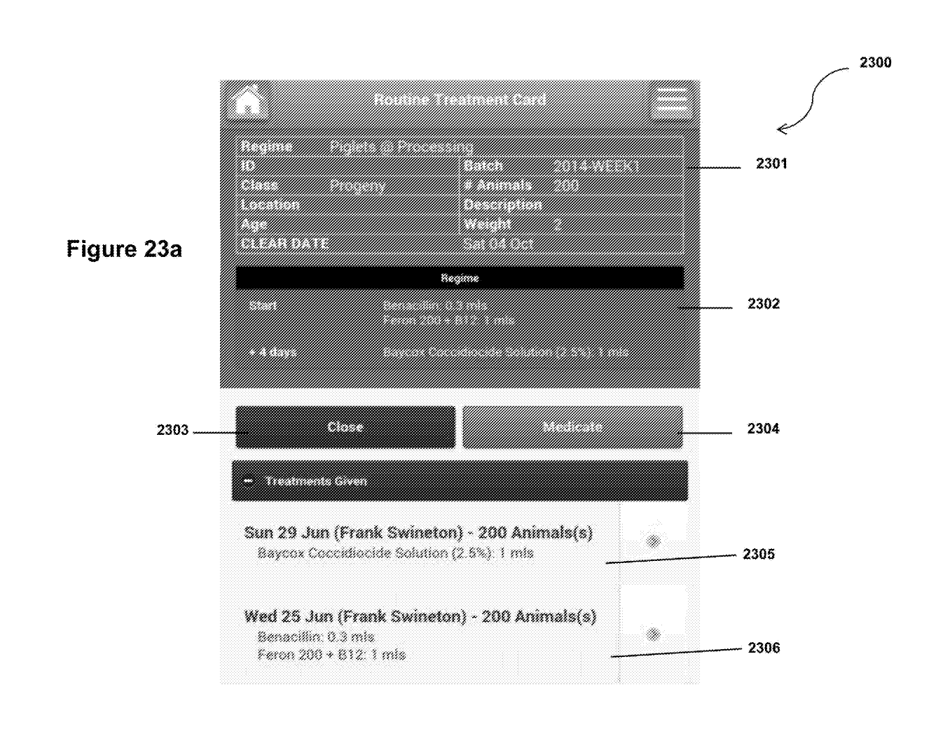

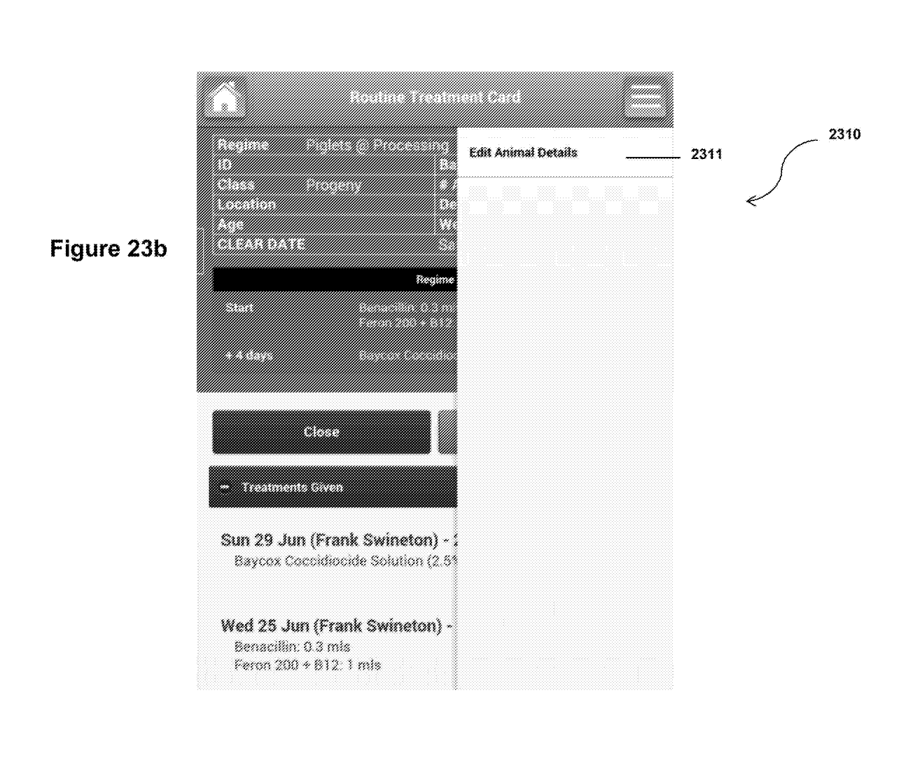

A Routine Treatment Regime may require further recording of treatment data. In this event, a user is able to locate the Routine Treatment Regime from either the Animals Requiring Attention List (see FIG. 17) if the treatment is due, or from a list of ongoing Routine Treatment Regimes (see 713 in FIG. 7b). This event results in the user being presented with the Routine Treatment Regimes treatment card (see FIG. 23a). The user is then able to commence entering additional treatment data via 2304. The user is presented with a form (see FIG. 22) where the due and upcoming treatments are listed 2202. They are able to select the medications to administer in a similar fashion to that described above when commencing the treatment. When the last treatment of a Routine Treatment Regime has been administered, the regime is automatically closed.

Routine Treatment Regimes that have finished can be viewed by selecting Ended Routine Treatments (see 715 in FIG. 7b).

Group Treatment is defined as a treatment plan involving the administration of a dose of medication per unit (e.g. body weight) for a population of animals. FIG. 3c is a flow diagram with corresponding example screen shots showing in further detail, the process for managing the health of one or more animals that require Group Treatment. At step 371 the user associated with user device 105, 110, 115 selects a Group Treatment Type. The user in this case may select between Preventive and Therapeutic Regimes 382.

Selection of one of the options, 371 moves to Step 372 in which the system determines Group Treatment Plans which may be applicable to that particular Type of Group Treatment as determined by type selected at step 371. Group Treatment Plans are automatically generated from the veterinary healthcare plan once the Group Treatment Type is provided. Group Treatment Regimes can be linked together and therefore create a sequence of Treatment Regimes to be scheduled for a batch of animals via the Approved Medication Planner (AML) (see 4814 and 4815 in FIG. 48).

At step 373, the user selects the appropriate Group Treatment Plan. The user in this example may select from the list of the following Group Treatment Plans: Grow-Finish Respiratory Disease Control, Weaner—Fallback Pigs 383. Control then moves to step 374 where the system 100 via user devices 105, 110, 115 determines the Group Treatment Plan. Control then moves to 375 which provide input screens 384 and 385 so as to capture measurement data about the group of animals to be treated, namely date and time, location, Batch ID, number of animals, number of morbid (dead) animals at time of treatment, average age and average weight of the group.

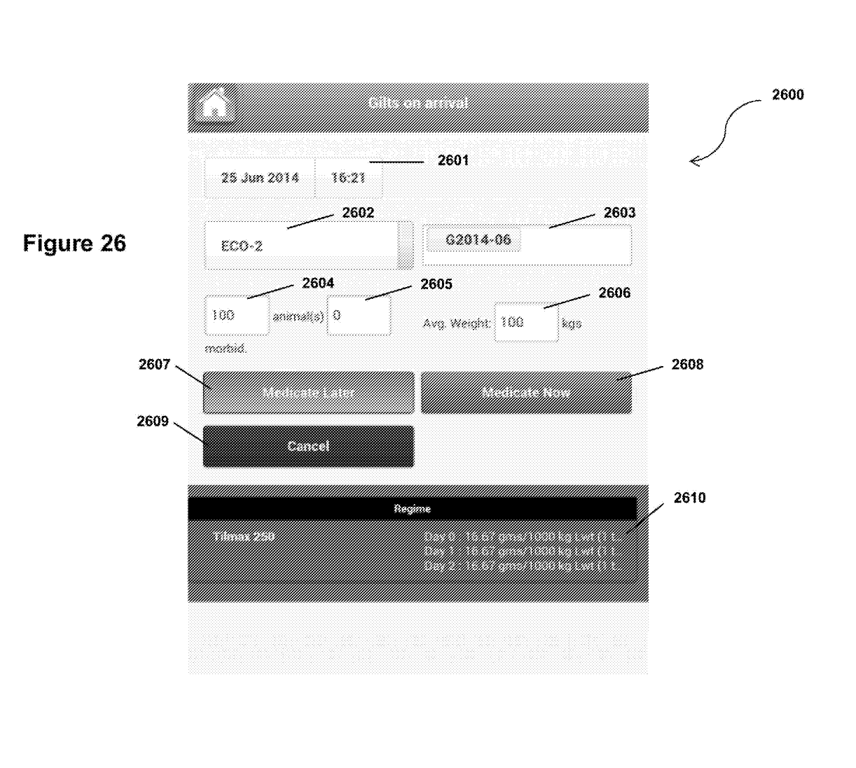

At step 376, the user indicates whether they wish to undertake the treatment immediately or at a later stage (see 2607 and 2708 in FIG. 26). A user may not be equipped to administer the required Group Treatment. The ‘medicate later’ option allows the system to identify the batch of animals and place the record on an action list to be treated later (see FIG. 17). If the user elects to carry out treatment at a later stage the method ends at 378.

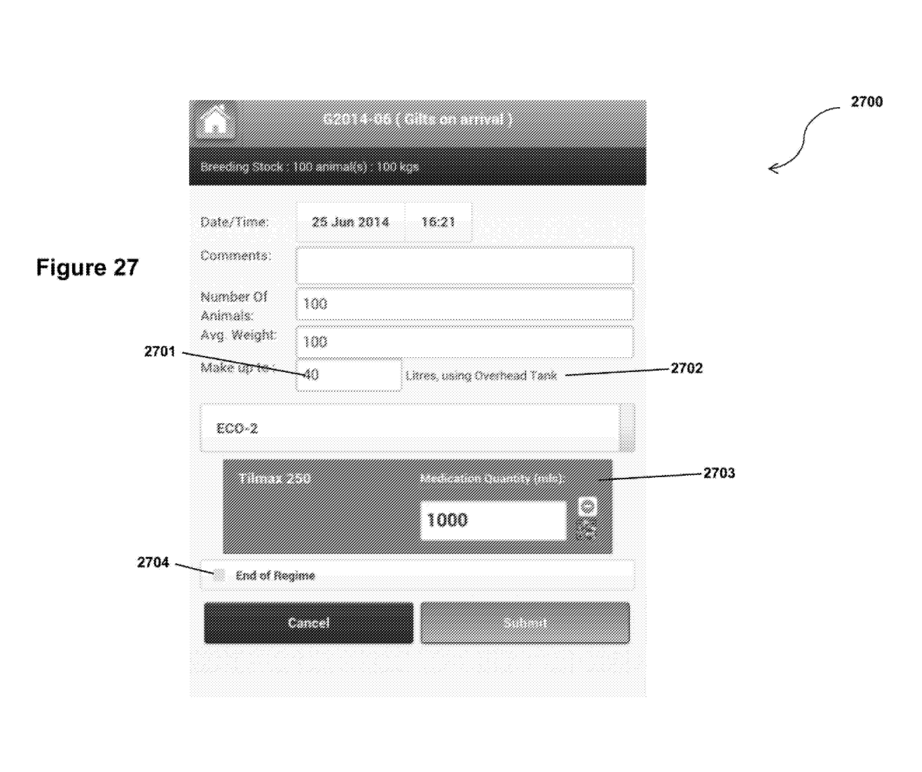

Otherwise if at step 376 the user elects to carry out treatment immediately, control moves to 379 where the system displays recommended prescribed treatments from a determined Group Treatment Regime (see 385 and 2703 in FIG. 27).

Screen shot 385 includes the date and time of treatment (this defaults to the current time, however this can be adjusted where retrospective data entry is conducted) and an option to provide comments. This screen specifies the Group Treatments as recommended by the healthcare professional for this particular Treatment Regime in addition to listing the other Treatment Regimes linked to this Treatment Regime (see FIG. 21). The user indicates which treatments are to be administered by using the [+/−] button 2703. If required, users may adjust the dose prescribed. The healthcare professional receives a notification where non-recommended doses are administered (see FIG. 44a). If a medication of the prescribed treatment plan is not administered, the invention displays a list of reasons from which the user selects the appropriate option (see FIG. 14c). Following the completion of this form, a notification is sent to the healthcare professional (see FIG. 44a).

The system 100 has provision for users to end a Group Treatment Regime prematurely (see FIG. 27).

Control then moves to step 380 in which the user indicates that the treatments have been administered. The user administering the treatment is selected from a list of users deemed competent. The system 100 may create an alert to follow up with the user following the treatment at a predetermined time after the treatment to help ensure subsequent treatments are administered as specified in the treatment plan and to determine whether or not the treatment was successful.

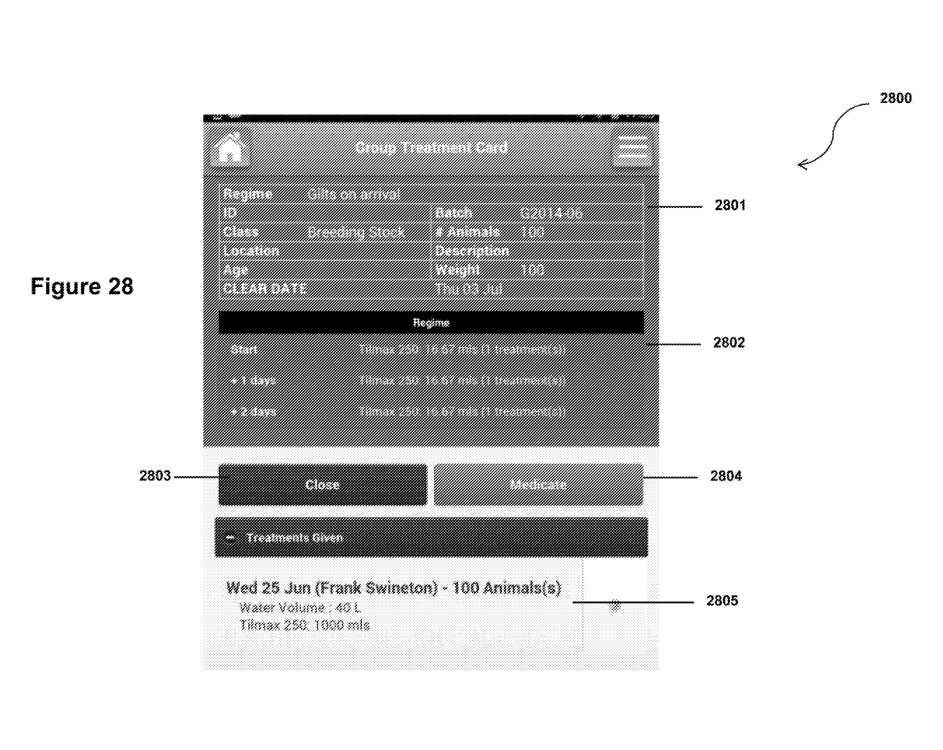

A Group Treatment Regime may require further recording of treatment data. In this event, a user is able to locate the Group Treatment Regime from either the Animals Requiring Attention List (see FIG. 17) if the treatment is due, or from a list of ongoing Group Treatment Regimes (see 714 in FIG. 7b). This event results in the user being presented with the Group Treatment Regimes treatment card (see FIG. 28). The user is then able to commence entering additional treatment data via 2804. The user is presented with a form (see FIG. 27) where the due and upcoming treatments are listed 2703. They are able to select the medications to administer in a similar fashion to that described above when commencing the treatment. When the last treatment of a Group Treatment Regime has been administered, the regime is automatically closed.

Group Treatment Regimes that have finished can be viewed by selecting Ended Group Treatments (see 716 in FIG. 7b).

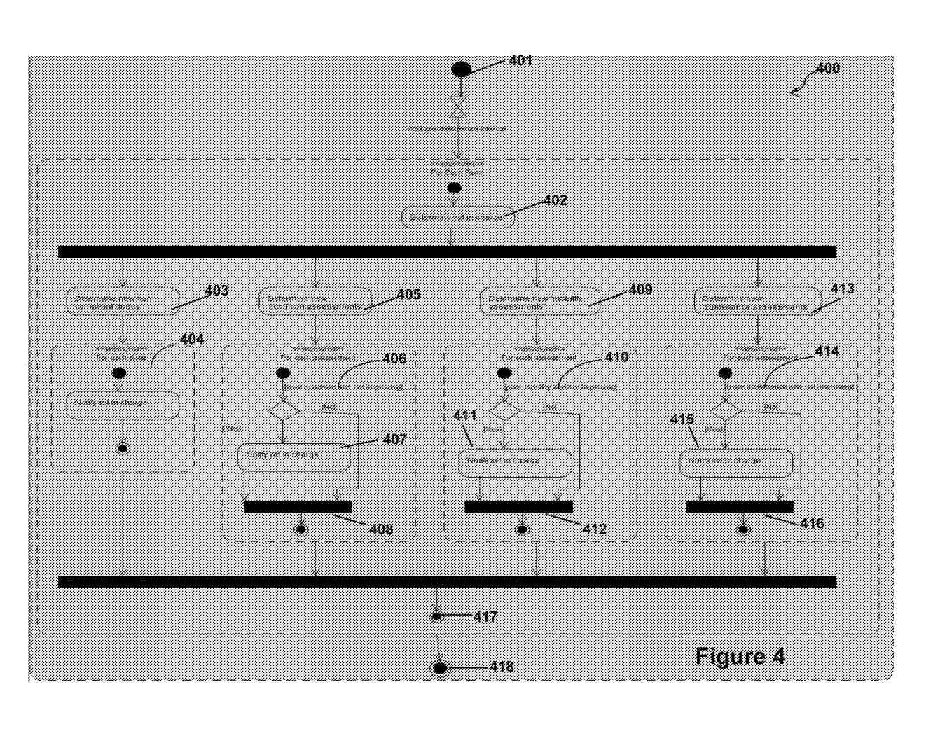

FIG. 4 is a flow diagram illustrating the notification of a healthcare professional following receipt of measurement data relating to the health or treatment of one or more animals in a remote location. At step 401 a predetermined wait interval is provided which relates to the interval in which the notification system waits before reviewing any new data. Preferably, this interval is initially set to every 5 minutes. At step 402 the system 100 determines the healthcare professional in charge of a particular location associated with the one or more animals. For example, a user associated with user device 105 may be associated with a healthcare professional associated with terminal 135 or 140 or 145, whereas a user associated with device 110 may be only associated with healthcare professionals associated with terminal 135. That is to say it may be that one or more healthcare professionals may be associated with one or more users which are located at a particular farm (for example).

The system 100 determines the healthcare professional in charge of each set of measurement data associated with animals or associated with a particular farm or location. At step 403 the system may notify the healthcare professional at terminal 135, 140, 145 based on whether or not there are any new non-compliant doses.

This is possible since the treatment plan specifies the dose. In the case of Individual and Group treatment, this dose is calculated from dose rate and the inputted body weight. If the dose administered varies from the recommended dose by more than a specified margin of error, then this is regarded as a non-compliant dose.

Control then moves to step 404 in which for each dose the healthcare professional is notified in the event of any non-compliant doses and control moves to step 417 which is the end of the notification loop for a client.

The system also routinely determines at step 405 whether or not any new “condition assessments” have occurred as determined from measurement data provided by the user in field 330 in FIG. 3a.

Control moves to step 406 in which for each set of measurement data it is determined whether or not the animal is in poor condition and not improving. If at step 406 it is determined that the animal is in poor condition and/or not improving, control moves to step 407 in which the healthcare professional associated with the user is notified via an alert.

At step 409 the system 100 also determines whether or not there are any new “mobility assessments” from the measurement data provided by the user 327 in FIG. 3a. Control then moves to step 410 where it is determined by the system 100 whether or not the new mobility assessments include mobility assessments which are poor and/or are not improving. In the event the measurement data indicates that there is poor mobility or no improvement, control moves to step 411 in which the healthcare professional is notified via an alert.

At step 413 the system 100 also determines whether or not there are any new “sustenance assessments”. In step 414 it is determined whether or not any of the new sustenance assessments are poor and/or are not improving. If at step 414 the new sustenance assessments are poor or not improving control moves to step 415 where the healthcare professional is notified via an alert.

After all processes relating to 403, 405, 409 and 413 are completed, control moves to step 417. If more farms exist, control moves to step 402. Otherwise if it is the last farm to process control moves to 418.

Step 418 is the end of the timing interval loop where the timing interval loop refers to the evaluation of all new data for all farms. From 418 control moves to step 401, where the notification process is re-initiated after the specified time period (for example 5 minutes) at 401.

It will be appreciated that steps 403, 405, 409 and 413 which determine non-compliant doses, condition assessments, mobility assessments and sustenance assessments may include other assessments as required such as remote sensing of body temperature, respiratory rate, heart rate, postural information and may notify a healthcare professional as appropriate.

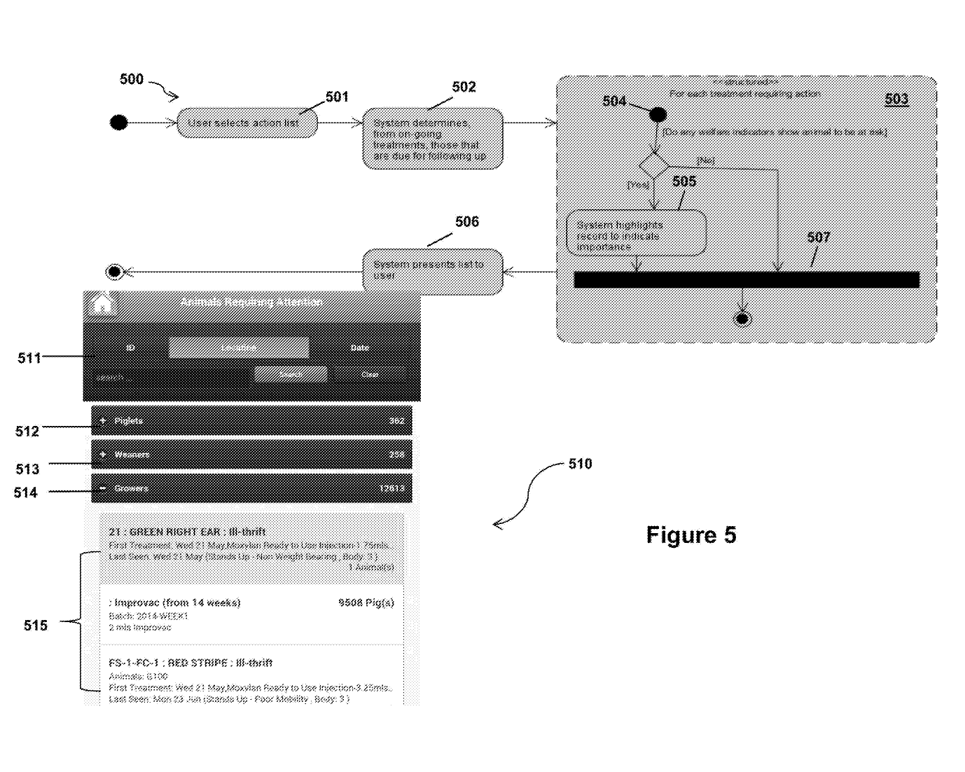

FIG. 5 is a flow diagram with a corresponding example screen shot (and FIG. 17) showing a status list of animals to a user associated with user devices 105, 110 and 115 of actions to be carried out based on the treatment data provided by the system 100 via the healthcare professional. At step 501 the user selects an action list and control moves to step 502 in which the system 100 determines, based on any ongoing treatments, which are due for follow up. Ongoing treatments for follow up may include: animals in treatment that have not been assessed within a specified time (this could be system wide, client specific, animal class specific or symptom specific), animals that are due for treatments based on their treatment plan, animals that are overdue for treatments based on their treatment plan. Control then moves to step 503 where it is determined whether any welfare indicators show any of the animals to be at risk. A welfare indicator may include not eating, not drinking, poor mobility, poor body condition and in time, abnormal body temperature, respiratory rate, heart rate and postural information for example.

At 504 it is determined whether or not welfare indicators show an animal to be at risk and if so control moves to step 505 where the system highlights a particular record to alert the user associated with user device 105, 110 or 115 to the animal perhaps requiring additional care. This is shown in screen shot FIG. 17 showing that Green Right Ear grower record located in pen 21 is highlighted as it has been assessed as non-weight bearing on 21 May. This provides the user with an alert that an animal may require more frequent assessment.

Control then moves to step 506 in which the system presents a list to the user and if the user takes action this is reported back to the server via measurement data.

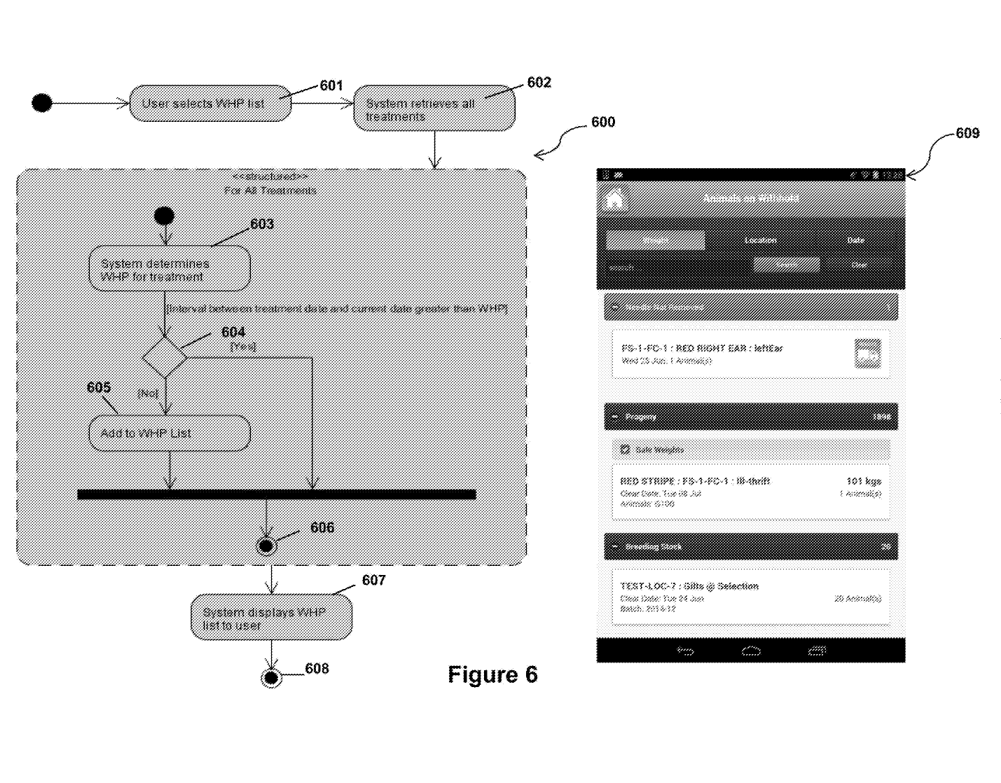

FIG. 6 is a flow diagram with a corresponding example screen shot (609 and FIG. 29) in which a user associated with terminal 105, 110 and 115 is alerted to animals (or their products such as milk) which are still on withhold from sale.

All products administered to animals (intended for human consumption) have a Withholding Period (WHP) and Export Slaughter Interval (ESI) which are approved by regulatory bodies. A withholding period for a product is defined by the amount of time needed to elapse between the last treatment of that product and the day it can be sold for human consumption. Complexities occur when an animal is treated with a number of different products at different times.

WHPs for each product-dose combination are specified in the system by the healthcare professional (see 4624 & 4625 in FIG. 46c).

In determining the WHP for an animal, the system 100, iterates through all treatments it has been given based by treatment date, product and dose to determine the date when the animal has complied with all WHPs/ESIs.

At step 601 the user associated with terminals 105, 110 and 115 may select a list of withheld animals (see 709 in FIG. 7a) at step 602 the system via the server 125 retrieves all of the treatments pending for that particular farm and at step 603 determines the withhold period for treatment.

Control then moves to step 604 where it is determined whether the interval between the treatment date and the present date is greater than a withhold (plus one day) period. In the event that the interval between the treatment date and current date is greater than the withhold period (plus one day), then the animal is safe for human use and control moves to step 606 which is the end point of the ‘For all treatments’ loop. From step 606, if more treatments exist, control moves to step 603 and the next treatment is assessed. Otherwise, control moves to 607 where the system displays a withhold period list of animals to the user and the method ends at step 608. If at step 604 it was determined that the interval between the treatment date and the present date is not greater than the withhold period (plus one day) then control moves to step 605 in which the animal is added to the withhold period list or maintained on the withhold period list (as the case may be) then control moves to step 606 and step 607 the system displays the current withhold period list to the user. This is best shown in FIG. 29 which illustrates for example that there is one 100 kg progeny pig in FS-1-FC-1 pen with a Red Stripe and with a unique animal identification tag of G100 which will be clear for sale on 8^{th} July.

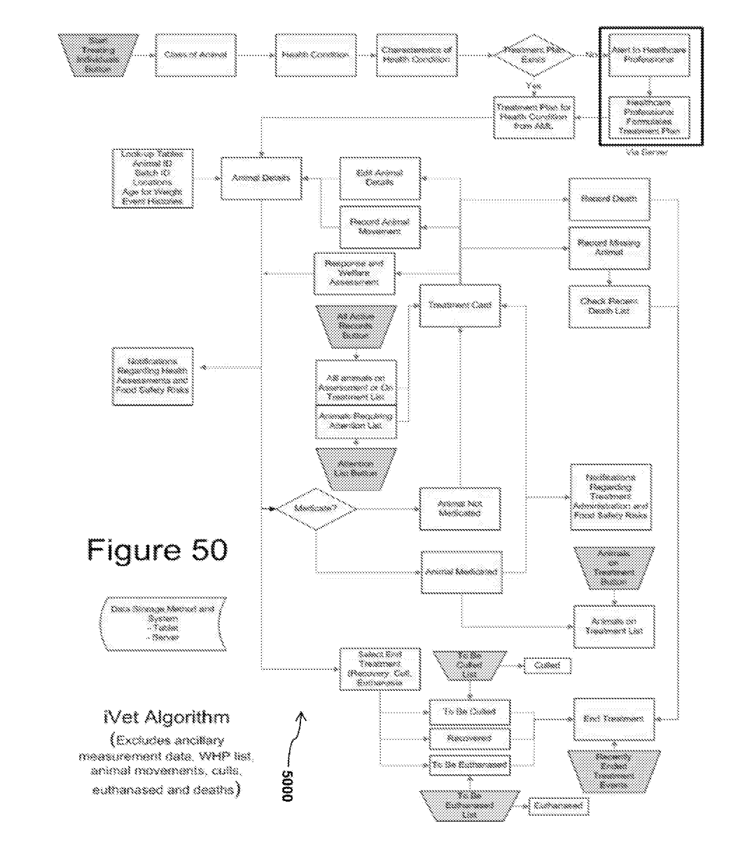

Referring now to FIG. 50, there is shown a flow chart of a method 5000 for remotely managing the health of animals. The method includes the steps receiving measurement data relating to the health or treatment of one or more animals in one or more remote locations over a network from a user device connected to the network. The user device includes a memory having predetermined treatment plans associated with predetermined measurement data. The measurement data is also stored at a server connected to the network. The user device compares the measurement data with the predetermined measurement data and provides treatment data indicative of a predetermined treatment plan associated with the predetermined measurement data. That is, after the characteristics of the health condition(s) have been determined by the user and inputted into the user device as measurement data, these characteristics are compared with predetermined treatment plans on the user device that have been previously designed by a healthcare professional.

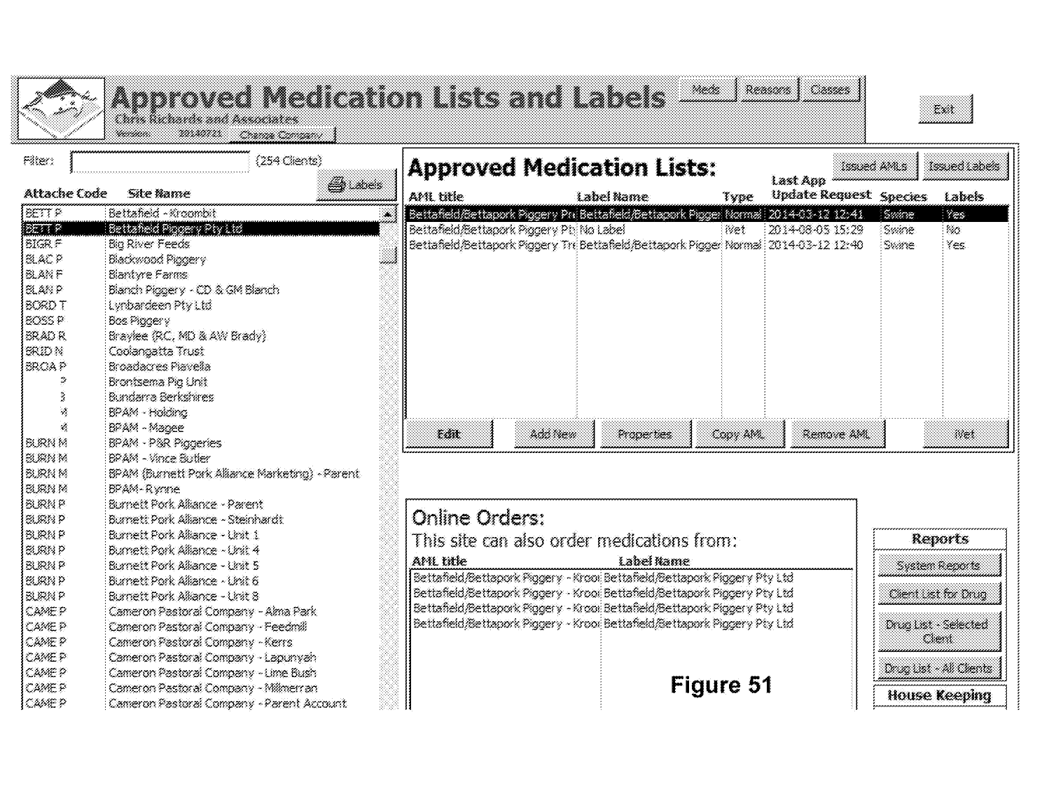

If a predetermined treatment plan cannot be matched, then an alert is sent to the health care professional along with measurement data to enable the design of a treatment plan for this animal on the server (see FIG. 51). Treatment plans on the server are synchronised with those on the user device. That is, the server compares the measurement data with one or more pre-determined criteria, contacts a healthcare professional via a device associated with the healthcare professional connected to the network for review of the measurement data if said one or more predetermined criteria are met and provides the measurement data to the device of the healthcare professional for review. The healthcare professional subsequently can provide a treatment plan.

The user follows the instructions of the treatment plan after the animal details have been entered, which includes a welfare assessment of the animal. However, if the user can delay the administration of treatment to a more convenient or appropriate time, this animal with be placed on the treatment task list. Animals entered are required to be reassessed for their welfare status e.g. daily. The animals due for reassessment are placed on the Animals Requiring Attention task list. Notifications are sent to health care professions of animals determined to be significantly compromised (based on comparison with predetermined criteria). Subsequent treatments required along with their instructions as defined in the treatment plan are placed into the Treatment Task List. Before subsequent treatments are given, the animal requires a welfare assessment. A health care professional is notified if animals have not received treatments as per the treatment plan or if they have been given incorrectly. This enables intervention by the health care professional which may include modifying the treatment plan, adjusting the animals withholding period, or determining disposition of the animal. That is, the healthcare professional subsequently provides a modified treatment plan of the predetermined treatment plan based on the measurement data and the server generates modified treatment data indicative of the modified treatment plan and stores the modified treatment data at the server. The modified treatment data is sent over the network to the user device for administration.

Once the treatment plan has been administered, the treatment is ended. The status of the treatment animal is determined when the treatment plan has been completed. Animals are deemed to have recovered, to be culled, to be euthanized or died. Treated animals are tracked explicitly by the system until they have completed their appropriate withholding period (e.g. when they are permitted to be sold for food consumption).

This method provides the ability to provide improved animal health both for individual and animal populations while ensuring the food safety risks associated with treated animals are appropriately managed and enable effective trace back and recalls should a problem occur. The method requires users to be allocated appropriate permissions, recording of medication batch numbers and expiry dates, animals to pass a fitness to load assessment prior to transport, and medication usage monitoring to assist with reducing the risk of the development of antibiotic resistant bacteria.

Further aspects of the method will be apparent from the above description of the system 100. Persons skilled in the art will also appreciate that the method could be embodied in program code. The program code could be supplied in a number of ways, for example on a tangible computer readable medium, such as a disc or a memory, or as a data signal or data file.

It is to be understood that various alterations, additions and/or modifications may be made to parts previously described without departing from the ambit of the present invention, and that, in the light of the above teachings, the present invention may be implemented in software, firmware and/or hardware in a variety of manners as would be understood by the person skilled in the art.

1. A method for remotely managing the health of animals, the method including:
receiving measurement data relating to the health or treatment of one or more animals in one or more remote locations over a network from a user device connected to the network;
storing the measurement data at a server connected to the network;
comparing the measurement data with one or more pre-determined criteria at the server;
contacting a healthcare professional via a device associated with the healthcare professional connected to the network for review of the measurement data if said one or more predetermined criteria are met;
providing the measurement data to the device of the healthcare professional for review and the healthcare professional subsequently providing a treatment plan based on the measurement data;
generating treatment data indicative of the treatment plan and storing the treatment data at the server; and
providing the treatment data over the network to the user device.

2. The method of claim 1, wherein the method further includes providing the measurement data to the device of the healthcare professional over the network for immediate review by a healthcare professional in real time.

3. The method of claim 1, wherein the method further includes receiving further measurement data from the user device relating to the health or treatment of said one or more animals following treatment according to the treatment plan, comparing the further measurement data with the treatment data at the server, and providing the further measurement data and the treatment data to the device of the health care professional over the network for review by the healthcare professional based on a result of the comparison.

4. The method of claim 1, wherein the method further including storing predetermined treatment data indicative of predetermined treatment plans provided by the healthcare professional based on predetermined measurement data.

5. The method of claim 4, wherein the method further includes comparing the measurement data with the predetermined measurement data at the server and providing the predetermined treatment data over the network to the user device if the measurement data corresponds to the predetermined measurement data.

6. The method of claim 5, wherein the method further includes providing the measurement data to the device of the healthcare professional for review if the measurement data does not correspond to the predetermined measurement data, and the healthcare professional subsequently providing a modified treatment plan based on the measurement data.

7. The method of claim 1, wherein the one or more pre-determined criteria include:
number of deaths within a class or symptom exceeding a threshold;
number of treatments within a class or symptom exceeding a threshold;
animals determined to be severely compromised as assessed by measurement data;
poor response to initial treatment plan;
inappropriate medication use as measured by medication product, dose, and frequency; or
the occurrence of an unknown health event.

8. The method of claim 1, wherein the measurement data relating to the health or treatment of one or more animals includes any one or more of:
a. pictures, video or text data, identification data;
b. the type of animal, the class of animal, date and time, location or number of animals;
c. a unique identifier associated with each animal or group of animals.

9. (canceled)

10. (canceled)

11. The method of claim 1, wherein
the treatment data includes prescribing a medication, indications for treatment, clinical response to treatment or a withholding period.

12. A system for remotely managing the health of animals, the system including:
a server for receiving and storing measurement data relating to the health or treatment of one or more animals in one or more remote locations over a network connected thereto from a user device connected to the network,
wherein the server is arranged to:
compare the measurement data with one or more pre-determined criteria and contact a healthcare professional via a device associated with the healthcare professional connected to the network for review of the measurement data if said one or more predetermined criteria are met;
provide the measurement data to the device of the healthcare professional for review and the healthcare professional subsequently providing a treatment plan based on the measurement data;
generate treatment data indicative of the treatment plan and to store the treatment data at the server; and
provide the treatment data over the network to the user device.

13. The system of claim 12, wherein the server is further arranged to provide the measurement data to the device of the healthcare professional over the network for immediate review by a healthcare professional in real time.

14. The system of claim 12, wherein the server is further arranged to receive further measurement data from the user device relating to the health or treatment of said one or more animals following treatment according to the treatment plan, compare the further measurement data with the treatment data at the server, and provide the further measurement data and the treatment data to the device of the health care professional over the network for review by the healthcare professional based on a result of the comparison.

15. The system of claim 12, wherein the server is further arranged to store predetermined treatment data indicative of predetermined treatment plans provided by the healthcare professional based on predetermined measurement data.

16. The system of claim 15, wherein the server is further arranged to compare the measurement data with the predetermined measurement data at the server and provide the predetermined treatment data over the network to the user device if the measurement data corresponds to the predetermined measurement data.

17. The system of claim 16, wherein the server is further arranged to provide the measurement data to the device of the healthcare professional for review if the measurement data does not correspond to the predetermined measurement data, and the healthcare professional subsequently providing a modified treatment plan based on the measurement data.

18. The system of claim 12, wherein the one or more pre-determined criteria include:
number of deaths within a class or symptom exceeding a threshold;
number of treatments within a class or symptom exceeding a threshold;
number of symptoms animals determined to be severely compromised as assessed by measurement data;
poor response to initial treatment plan;
inappropriate medication use as measured by medication product, dose, and frequency; or
the occurrence of an unknown health event.

19. The system of claim 12, wherein the measurement data relating to the health or treatment of one or more animals includes any one or more of:
pictures, video or text data, identification data;
the type of animal, the class of animal, date and time, location or number of animals; and
a unique identifier associated with each animal or group of animals.

20. (canceled)

21. (canceled)

22. The system of claim 12, wherein the treatment data includes prescribing a medication, indications for treatment, clinical response to treatment or a withholding period.

23. A method for remotely managing the health of animals, the method including:
receiving measurement data relating to the health or treatment of one or more animals in one or more remote locations over a network from a user device connected to the network, wherein the user device includes a memory having predetermined treatment plans associated with predetermined measurement data
storing the measurement data at a server connected to the network;
the user device comparing the measurement data with the predetermined measurement data and providing treatment data indicative of a predetermined treatment plan associated with the predetermined measurement data;
the server comparing the measurement data with one or more pre-determined criteria;
contacting a healthcare professional via a device associated with the healthcare professional connected to the network for review of the measurement data if said one or more predetermined criteria are met;
providing the measurement data to the device of the healthcare professional for review and the healthcare professional subsequently providing a modified treatment plan of the predetermined treatment plan based on the measurement data;
generating modified treatment data indicative of the modified treatment plan and storing the modified treatment data at the server; and
providing the modified treatment data over the network to the user device.

24. (canceled)
